(19) United States
(12) Patent Application Publication
(10) Pub. No.: US 2010/0273835 A1
(43) Pub. Date: Oct. 28, 2010

(54) NOVEL COMPOUNDS

(75) Inventors: Ulrich Heiser, Halle/Saale (DE); Andre J. Niestroj, Sennewitz (DE); Ingo Schulz, Halle/Saale (DE)

Correspondence Address: SONNENSCHEIN NATH & ROSENTHAL LLP, P.O. BOX 061080, WACKER DRIVE STATION, WILLIS TOWER, CHICAGO, IL, 60606-1080, US

(73) Assignee: PROBIODRUG AG, Halle/Saale (DE)

(21) Appl. No.: 12/665,502

(22) PCT Filed: Jul. 10, 2008
(86) PCT No.: PCT/EP2008/058977

§ 371 Date: Jun. 28, 2010

Related U.S. Application Data
(60) Provisional application No. 60/948,730, filed on Jul. 10, 2007.

(51) Int. Cl. A61K 31/4402; C07D 277/68; A61K 31/428; C07D 333/38; A61K 31/381; C07D 277/56; A61K 31/427; C07D 213/78; C07D 307/68; A61K 31/341; C07D 403/06; A61K 31/404; A61P 25/28; A61P 25/16; A61P 25/18; A61P 25/24; A61P 9/00; A61P 7/02

(52) U.S. Cl. 514/354; 548/180; 514/367; 549/72; 514/448; 548/200; 514/365; 546/328; 549/488; 514/471; 548/467; 514/419

(57) ABSTRACT

The invention provides compounds of general formula (I)

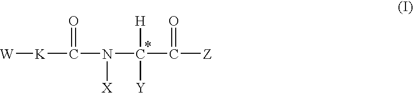

or a pharmaceutically acceptable salt, polymorph or solvate thereof, including all tautomers and stereoisomers thereof, wherein K, W, X; Y and Z are described throughout the description and claims.

The compounds of the present invention are useful as inhibitors of prolyl endopeptidase (PEP, EC 3.4.21.26) and/or IL-6.

### FIELD OF THE INVENTION

This invention relates to heteroaryl-carbonyl compounds as inhibitors of prolyl endopeptidase (PEP, EC 3.4.21.26) and/or IL-6 and/or effectors of Abeta.

### BACKGROUND OF THE INVENTION

Prolyl endopeptidase (PEP; EC 3.4.21.26; also called prolyl oligopeptidase) is a serine peptidase characterized by oligopeptidase activity. It is the name given to enzymes of family S9A, prolyl oligopeptidases, in clan SC (1). Enzymes belonging to clan SC are distinct from trypsin- or subtilisin-type serine peptidases by structure and by order of the catalytic triad residues in the primary sequence (2;3). The three dimensional structure of PEP revealed a two domain organization (4). The catalytic domain displays an α/β hydrolase fold in which the catalytic triad (Ser554, His680, Asp641) is covered by a so-called β-propeller domain. Most likely, the propeller domain controls the access of potential substrates to the active site of the enzyme and excludes peptides having more than 30 amino acids.

In contrast to the profound knowledge of the enzymatic and structural properties of PEP, the biological function of this enzyme is far from being fully understood (5;6). PEP is highly conserved in mammals and is ubiquitously distributed, with high concentrations occurring in the brain (7). Recently, the enzyme gained pharmaceutical interest due to a reported cognitive enhancement induced by treatment with specific PEP inhibitors. In rats displaying scopolamine-induced amnesia, PEP inhibition caused acetylcholine release in the frontal cortex and hippocampus (8). Furthermore, administration of a PEP inhibitor in rats with middle cerebral artery occlusion prolonged passive avoidance latency and reduced the prolonged escape latency in the Morris water maze task (9). The potential of PEP inhibitors as antidementia drugs was further confirmed by reports of neuroprotective effects. Inducing neurodegeneration in cerebellar granule cells led to increased neuronal survival and enhanced neurite outgrowth in presence of a PEP inhibitor (10). Moreover, the level of m₃-muscarinic acetylcholine receptor mRNA was found to be increased after PEP inhibition. This resulted in a stimulated phosphoinositide turnover.

It has been hypothesized that these effects are due to modulation of neuropeptide bioactivity by PEP (11). In vitro, PEP is able to rapidly inactivate several neuropeptides, including substance P and arginine-vasopressin (AVP) by limited proteolysis (12;13). Neuropeptides, such as substance P or AVP are known to influence learning and memory (14;15). The administration of substance P can induce long-term potentiation (LTP), a well established parameter for learning and memory (16). Binding of substance P to neurokinin 1 receptor stimulates a G-protein mediated increase in IP₃ concentration and a release of Ca²⁺ from intracellular stores within the endoplasmic reticulum (ER) (17;18). It is well established, but untested for substance P, that Ca²⁺ release from these stores is implicated in the induction of LTP and in learning and memory (19). In postsynaptic cells, LTP is prevented by the inhibition of IP₃ receptors, demonstrating the crucial role of IP₃ formation and Ca²⁺ release in this learning and memory model (20). It should be noted, however, that PEP is primarily located in the cytosol (21), whereas the interaction between the neuropeptides and their receptors takes place on the cell surface. Recently, Hasebe et al. found, that cytosolic prolyl endopeptidase is involved in the degradation of p40-phox splice variant protein in myeloid cells (22).

EP 0 172 458 discloses N-phenyl alkanoyl pyrrolidine derivatives useful as anti-amnesic agents.

EP 0 359 547 discloses pyridine compounds inhibiting prolylendo peptidase activity and useful for the treatment of amnesia.

U.S. Pat. No. 5,340,832 discloses N-substituted carbamoyl-alkanoyl-prolinal derivatives useful as inhibitors of prolyl endopeptidase for treating amnesia.

U.S. Pat. No. 5,763,576 discloses tetrapeptide alpha-ketoamides as selective and total inhibitors of serine and cysteine proteases. These compounds are useful in the treatment of tissue damage and various inflammatory conditions, such as blistering, and in the treatment of neurodegenerative diseases such as ischemia, stroke and Alzheimer's disease. The compounds are also inhibitors for blood coagulation enzymes and are useful anticoagulants for the treatment of thrombosis.

WO 91/18891 discloses aromatic pyrrolidine and thiazolidine amide(s) as prolyl endopeptidase inhibitors, which are useful for treating CNS disorders such as various memory or learning dysfunctions associated with disease e.g. Alzheimer's disease; amnesia; dementia; anxiety; ischemia; and damage caused by stroke.

WO 94/12474 discloses cyclic ketone compounds as prolyl endopeptidase inhibitors—including two nitrogen-containing heterocycles linked by a carbonyl group. These compounds inhibit the degradation and deactivation of TRH, substance P, neurotensin and vasopressin. They are useful for the treatment and prevention of amnesia and of dementia including Alzheimer's disease.

WO 95/03277 discloses N-substituted pyrrolidinyl-oxo-acetamide compounds as protease (especially PEP) inhibitors useful for treating memory loss e.g. Alzheimer's disease, and auto-immune disorders.

WO 95/15310 discloses prolyl peptide derivatives as prolyl endopeptidase inhibitors. These compounds can be used as memory enhancing agents to improve mental capacity, ability to recall cognitive events, and learned motor activities. Thus the compounds of WO 95/15310 may be used in patients suffering from aphasia, apraxia, agnosia, or any type of amnesias, benign forgetfulness and Korsakoff's syndrome. The compounds may also be used to prevent or slow memory deficits.

WO 97/07116 discloses PEP inhibitors for the use in treatment of acute events (such as ischemia and hypoxia) and progressive neurodegenerative disorders, including Alzheimer's disease, AIDS dementia and Huntington's disease.

WO 98/35960 discloses PEP inhibitors useful as nootropics having memory enhancing and anti-amnesic effects useful in the treatment of age-related cognitive decline and neuroprotectants useful for treatment of acute events (ischemia/hypoxia) and progressive neurodegenerative disorders such as Alzheimer's disease, AIDS related dementia and Huntington's disease.

WO 00/09542 discloses alpha-keto heterocycles inhibiting the enzymatic activity of a serine proteases. The compounds can be used to inhibit microbial growth, reduce perioperative blood loss, preserve transplantation tissues or organs, inhibit cancer cell growth or tumor progression or tumor metastasis or invasion, treat pulmonary vascular disease, restenosis or pulmonary hypertension myocarditis, bronchopulmonary dysplasia, myocardial necrosis or post-cardiac transplant coronary arteriopathy, atherosclerosis, reperfusion injury, Alzheimer's disease, hypoxia, ischemia and blood coagulation disorders.

U.S. Pat. No. 5,547,978 discloses PEP inhibitors based on pyrrolidin-2-ylcarbonylheterocyclic compounds, they can be used to inhibit PEP in mammalian brain for a pharmaceutical effect.

US2005/0171112 discloses the PEP inhibitor ZW215 of the formula

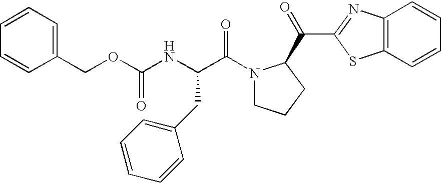

### DEFINITIONS

The term “PEP-inhibitor” or “prolyl endopeptidase inhibitor” is generally known to a person skilled in the art and means enzyme inhibitors which inhibit the catalytic activity of prolyl endopeptidase (PEP, prolyl oligopeptidase, POP).

“PEP activity” is defined as the catalytic activity of an endoprotease that is capable to hydrolyze post proline bonds in peptides or proteins where the proline is in amino acid position 3 or higher counted from the N-terminus of a peptide or protein substrate. “PEP-like enzymes” are enzymatically active proteins or peptides, which have PEP activity and are thereby inhibited by PEP-inhibitors.

The term “IL-6-inhibitor” is generally known to a person skilled in the art and means the reduction of the IL-6-level in a defined cell-system in correlation to an untreated control sample.

The term “effectors for Abeta” means the enhancement of the Abeta-level in a defined cell-system in correlation to an untreated control sample.

As used herein, the term “pharmaceutically acceptable” embraces both human and veterinary use: for example the term “pharmaceutically acceptable” embraces a veterinarily acceptable compound or a compound acceptable in human medicine and health care.

Throughout the description and the claims the expression “alkyl”, unless specifically limited, denotes a C₁₋₁₂ alkyl group, suitably a C₁₋₆ alkyl group, e.g. C₁₋₄ alkyl group. Alkyl groups may be straight chain or branched. Suitable alkyl groups include, for example, methyl, ethyl, propyl (e.g. n-propyl and isopropyl), butyl (e.g. n-butyl, iso-butyl, sec-butyl and tert-butyl), pentyl (e.g. n-pentyl), hexyl (e.g. n-hexyl), heptyl (e.g. n-heptyl) and octyl (e.g. n-octyl). The expression “alk”, for example in the expressions “alkoxy”, “haloalkyl” and “thioalkyl” should be interpreted in accordance with the definition of “alkyl”. Exemplary alkoxy groups include methoxy, ethoxy, propoxy (e.g. n-propoxy), butoxy (e.g. n-butoxy), pentoxy (e.g. n-pentoxy), hexoxy (e.g. n-hexoxy), heptoxy (e.g. n-heptoxy) and octoxy (e.g. n-octoxy). Exemplary thioalkyl groups include methylthio-. Exemplary haloalkyl groups include fluoroalkyl e.g. CF₃.

The expression “alkenyl”, unless specifically limited, denotes a C₂₋₁₂ alkenyl group, suitably a C₂₋₆ alkenyl group, e.g. a C₂₋₄ alkenyl group, which contains at least one double bond at any desired location and which does not contain any triple bonds. Alkenyl groups may be straight chain or branched. Exemplary alkenyl groups including one double bond include propenyl and butenyl. Exemplary alkenyl groups including two double bonds include pentadienyl, e.g. (1E,3E)-pentadienyl.

The expression “alkynyl”, unless specifically limited, denotes a C₂₋₁₂ alkynyl group, suitably a C₂₋₆ alkynyl group, e.g. a C₂₋₄ alkynyl group, which contains at least one triple bond at any desired location and may or may not also contain one or more double bonds. Alkynyl groups may be straight chain or branched. Exemplary alkynyl groups include propynyl and butynyl.

The expression “alkylene” denotes a chain of formula —(CH₂)ₙ— wherein n is an integer e.g. 1-6, unless specifically limited.

The expression “cycloalkyl”, unless specifically limited, denotes a C₃₋₁₀ cycloalkyl group (i.e. 3 to 10 ring carbon atoms), more suitably a C₃₋₈ cycloalkyl group, e.g. a C₃₋₆ cycloalkyl group. Exemplary cycloalkyl groups include cyclopropyl, cyclobutyl, cyclopentyl, cyclohexyl, cycloheptyl and cyclooctyl. A most suitable number of ring carbon atoms is three to six.

The expression “cycloalkenyl”, unless specifically limited, denotes a C₅₋₁₀ cycloalkenyl group (i.e. 5 to 10 ring carbon atoms), more suitably a C₅₋₈ cycloalkenyl group e.g. a C₅₋₆ cycloalkenyl group. Exemplary cycloalkenyl groups include cyclopropenyl, cyclohexenyl, cycloheptenyl and cyclooctenyl. A most suitable number of ring carbon atoms is five to six.

The expression “carbocyclyl”, unless specifically limited, denotes any ring system in which all the ring atoms are carbon and which contains between three and twelve ring members, suitably between three and ten ring members and more suitably between three and eight ring members. Carbocyclyl groups may be saturated or partially unsaturated, but do not include aromatic rings. Examples of carbocyclyl groups include monocyclic, bicyclic, and tricyclic ring systems, in particular monocyclic and bicyclic ring systems. Other carbocylcyl groups include bridged ring systems (e.g. bicyclo[2,2,1]heptenyl). A specific example of a carbocyclyl group is a cycloalkyl group. A further example of a carbocyclyl group is a cycloalkenyl group.

The expression “heterocyclyl”, unless specifically limited, refers to a carbocyclyl group wherein one or more (e.g. 1, 2 or 3) ring atoms are replaced by heteroatoms selected from N, S and O. A specific example of a heterocyclyl group is a cycloalkyl group (e.g. cyclopentyl or more particularly cyclohexyl) wherein one or more (e.g. 1, 2 or 3, particularly 1 or 2, especially 1) ring atoms are replaced by heteroatoms selected from N, S or O. Exemplary heterocyclyl groups containing one hetero atom include pyrrolidine, tetrahydrofuran and piperidine, and exemplary heterocyclyl groups containing two hetero atoms include morpholine and piperazine. A further specific example of a heterocyclyl group is a cycloalkenyl group (e.g. a cyclohexenyl group) wherein one or more (e.g. 1, 2 or 3, particularly 1 or 2, especially 1) ring atoms are replaced by heteroatoms selected from N, S and O. An example of such a group is dihydropyranyl (e.g. 3,4-dihydro-2H-pyran-2-yl-).

The expression “aryl”, unless specifically limited, denotes a C₆₋₁₂ aryl group, suitably a C₆₋₁₀ aryl group, more suitably a C₆₋₈ aryl group. Aryl groups will contain at least one aromatic ring (e.g. one, two or three rings). An example of a typical aryl group with one aromatic ring is phenyl. An example of a typical aryl group with two aromatic rings is naphthyl.

The expression “heteroaryl”, unless specifically limited, denotes an aryl residue, wherein one or more (e.g. 1, 2, 3, or 4, suitably 1, 2 or 3) ring atoms are replaced by heteroatoms selected from N, S and O, or else a 5-membered aromatic ring containing one or more (e.g. 1, 2, 3, or 4, suitably 1, 2 or 3) ring atoms selected from N, S and O. Exemplary monocyclic heteroaryl groups having one heteroatom include: five membered rings (e.g. pyrrole, furan, thiophene); and six membered rings (e.g. pyridine, such as pyridin-2-yl, pyridin-3-yl and pyridin-4-yl). Exemplary monocyclic heteroaryl groups having two heteroatoms include: five membered rings (e.g. pyrazole, oxazole, isoxazole, thiazole, isothiazole, imidazole, such as imidazol-1-yl, imidazol-2-yl imidazol-4-yl); six membered rings (e.g. pyridazine, pyrimidine, pyrazine). Exemplary monocyclic heteroaryl groups having three heteroatoms include: 1,2,3-triazole and 1,2,4-triazole. Exemplary monocyclic heteroaryl groups having four heteroatoms include tetrazole. Exemplary bicyclic heteroaryl groups include: indole (e.g. indol-6-yl), benzofuran, benzthiophene, quinoline, isoquinoline, indazole, benzimidazole, benzthiazole, quinazoline and purine.

The expression “-alkylaryl”, unless specifically limited, denotes an aryl residue which is connected via an alkylene moiety e.g. a C₁₋₄alkylene moiety.

The expression “-alkylheteroaryl”, unless specifically limited, denotes a heteroaryl residue which is connected via an alkylene moiety e.g. a C₁₋₄alkylene moiety.

The term “halogen” or “halo” comprises fluorine (F), chlorine (Cl) bromine (Br) and iodine (I).

The term “amino” refers to the group —NH₂.

The term “amino acid side chain” or “side chain of an amino acid” refers to the characteristic side moiety R of an amino acid RCH(NH₂)COOH. For example, the side chain of phenylalanine (Phe) is —CH₂Ph.

Stereoisomers:

All possible stereoisomers of the claimed compounds are included in the present invention. Where the compounds according to this invention have at least one chiral center, they may accordingly exist as enantiomers. Where the compounds possess two or more chiral centers, they may additionally exist as diastereomers. It is to be understood that all such isomers and mixtures thereof are encompassed within the scope of the present invention.

Pharmaceutically Acceptable Salts:

In view of the close relationship between the free compounds and the compounds in the form of their salts, whenever a compound is referred to in this context, a corresponding salt is also intended, provided such is possible or appropriate under the circumstances. The pharmaceutically acceptable salt may take a form in which a basic side chain is protonated with an inorganic or organic acid. Representative organic or inorganic acids include hydrochloric, hydrobromic, perchloric, sulfuric, nitric, phosphoric, acetic, propionic, glycolic, lactic, succinic, maleic, fumaric, malic, tartaric, citric, benzoic, mandelic, methanesulfonic, hydroxyethanesulfonic, benzenesulfonic, oxalic, pamoic, 2-naphthalenesulfonic, p-toluenesulfonic, cyclohexanesulfamic, salicylic, saccharinic or trifluoroacetic acid. Alternatively it may take the form in which an acidic side chain forms a salt with a metal ion (e.g. sodium, potassium ions and the like) or other positive ion such as ammonium. All pharmaceutically acceptable acid addition salt forms of the compounds of the present invention are intended to be embraced by the scope of this invention.

Polymorph Crystal Forms and Solvates:

Furthermore, some of the crystalline forms of the compounds may exist in more than one polymorphic form and as such all forms are intended to be included in the present invention. In addition, some of the compounds may form solvates with water (i.e. hydrates) or common organic solvents, and such solvates are also intended to be encompassed within the scope of this invention. The compounds, including their salts, can also be obtained in the form of their hydrates, or include other solvents used for their crystallization.

Prodrugs:

The present invention further includes within its scope prodrugs of the compounds of this invention. In general, such prodrugs will be functional derivatives of the compounds which are readily convertible in vivo into the desired therapeutically active compound. Thus, in these cases, the methods of treatment of the present invention, the term “administering” shall encompass the treatment of the various disorders described with prodrug versions of one or more of the claimed compounds, but which converts to the above specified compound in vivo after administration to the subject. Conventional procedures for the selection and preparation of suitable prodrug derivatives are described, for example, in “Design of Prodrugs”, ed. H. Bundgaard, Elsevier, 1985 and the patent applications DE 198 28 113, DE 198 28 114, WO 99/67228 and WO 99/67279 which are fully incorporated herein by reference.

As used herein, the term “composition” is intended to encompass a product comprising the claimed compound(s) in the therapeutically effective amounts, as well as any product which results, directly or indirectly, from combinations of the claimed compounds.

### BRIEF DESCRIPTIONS OF THE DRAWINGS

FIG. 1: Quantification of basal medium IL-6 in human glial U-343 cells treated with different PEP inhibitors.

FIG. 2: Quantification of basal Abeta 1-42 value in human neuroblastoma SH-SY5Y cells treated with different PEP inhibitors.

### SUMMARY OF THE INVENTION

According to the invention there are provided compounds of formula (I),

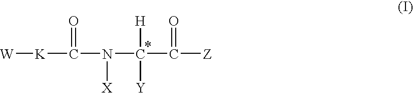

or a pharmaceutically acceptable salt, polymorph or solvate thereof, including all tautomers and stereoisomers thereof,

wherein:

K represents O, S or NH;

W represents —C₁₋₆alkyl-aryl, —C₂₋₆alkenylaryl; —C₁₋₆alkylheteroaryl or —C₂₋₆alkenylheteroaryl;

X represents H or methyl;

Y represents the side chain of an amino acid selected from

Gly; Ala; Val; Leu; Ile; Met; Phe; Ser; Thr; Trp; Asn; Gln; and

the side chain of an analogue of Phe in which the aromatic moiety is substituted by one more groups selected from halogen, nitro, C₁₋₄alkyl, C₁₋₄haloalkyl, hydroxyl, C₁₋₄alkoxy and C₁₋₄haloalkoxy; and

the side chain of an analogue of Ser or Thr in which the hydroxyl group is substituted by C₁₋₆alkyl; and

the side chain of an analogue of Trp in which the heteroaromatic moiety is substituted by one or more C₁₋₄alkyl groups; and

the side chain of an analogue of Cys in which the thiol group is substituted by C₁₋₆alkyl; and

the side chain of an analogue of Asp or Glu wherein the carboxylic acid group has been converted into a C₁₋₆alkyl ester; and

the side chain of an analogue of Asn or Gln wherein the —NH₂ of the amide has been converted into an —NH(C₁₋₄alkyl) or —N(C₁₋₄alkyl)(C₁₋₄alkyl) group; and

the side chain of an analogue of Lys or Arg wherein the —NH₂ of the amine has been converted into an —NHC(O)C₁₋₄alkyl group or an —N(C₁₋₄alkyl)C(O)C₁₋₄alkyl group;

or X and Y are joined such that

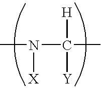

represents

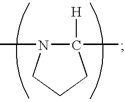

Z represents heteroaryl;

and

when Y represents the side chain of an amino acid selected from Phe; Trp; and

the side chain of an analogue of Phe in which the aromatic moiety is substituted by one more groups selected from halogen, nitro, C₁₋₄alkyl, C₁₋₄haloalkyl, hydroxyl, C₁₋₄alkoxy and C₁₋₄haloalkoxy; and

the side chain of an analogue of Trp in which the heteroaromatic moiety is substituted by one or more C₁₋₄alkyl groups;

then Z can also represent aryl;

wherein any of the aforesaid carbocyclyl and heterocyclyl may be optionally substituted by one or more groups selected from oxo and methyl; and

wherein any of the aforesaid aryl and heteroaryl may optionally substituted by one or more groups selected from: C₁₋₆alkyl, C₂₋₆alkenyl, C₂₋₆alkynyl, C₁₋₆haloalkyl, —C₁₋₆thioalkyl, —SO₂C₁₋₄alkyl, C₁₋₆alkoxy-, —O—C₃₋₈cycloalkyl, C₃₋₈cycloalkyl, —SO₂C₃₋₈cycloalkyl, C₃₋₆alkenyloxy-, C₃₋₆alkynyloxy-, —C(O)C₁₋₆alkyl, —C(O)OC₁₋₆alkyl, C₁₋₆alkoxy-C₁₋₆alkyl-, nitro, halogen, cyano, hydroxyl, —C(O)OH, —NH₂, —NHC₁₋₄alkyl, —N(C₁₋₄alkyl)(C₁₋₄alkyl), —C(O)N(C₁₋₄alkyl)(C₁₋₄alkyl), —C(O)NH₂ and —C(O)NH(C₁₋₄alkyl);

wherein * represents a stereogenic centre;

and wherein the following compounds (a) to (k) are disclaimed from the definition of formula (I):

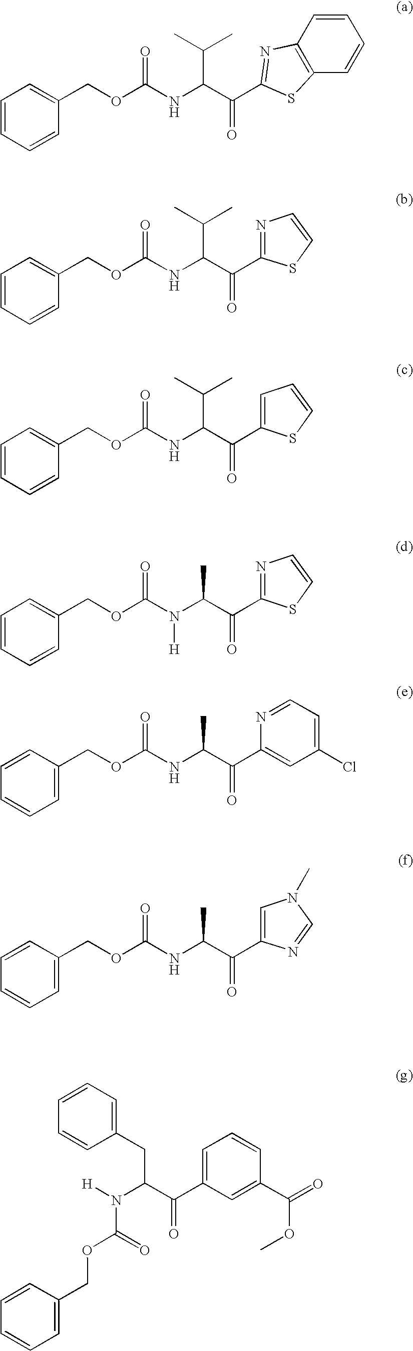

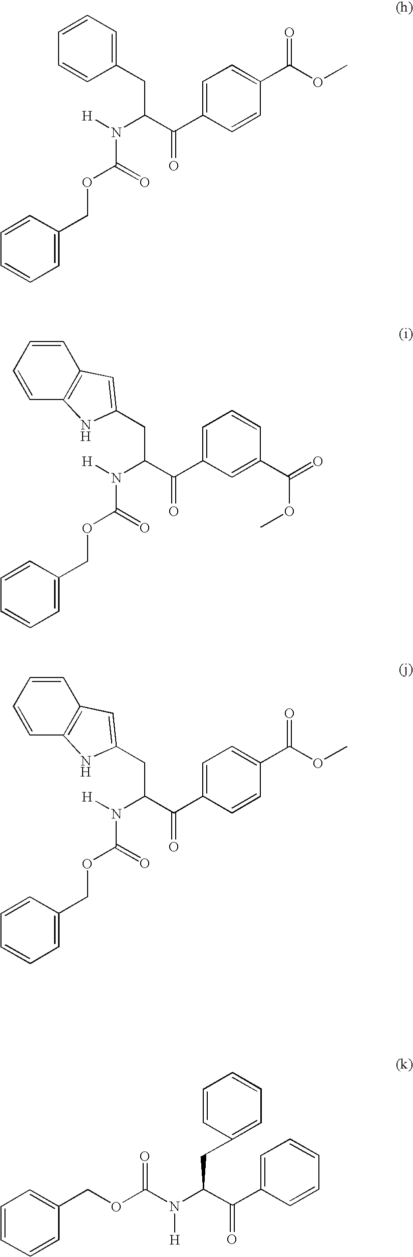

Compounds (a), (b) and (c) are disclosed by KYOWA HAKKO KOGYO in WO 2006/006644 A.

Compound (d) is disclosed by Ali, A. et al. (2006) in WO 2006/014413 and by Conrad, K. et al. ((2005) Tetrah. Lett. 46, 8587-8589);

Compounds (e) and (f) are disclosed by Ali, A. et al. (2006) in WO 2006/014413.

Compounds (g), (h) (i) and (j) are disclosed by SANKYO CO LTD in JP2001131137 (2001).

Compound (k) is disclosed by Dixon, D et al. ((2004) Org. Lett. 6, 4423-4426), Tokuyama, H. et al. ((1998) J. Braz. Chem. Soc. 9, 381-387) and Tokuyama, H. et al. ((1998) Tetrah. Lett. 39, 3189-3192).

Compounds (a) to (k) are disclosed in the aforementioned documents as chemical intermediates with no pharmaceutical activity being referred to.

We also provide Examples 11 to 26 which fall outside of formula (I) above.

The aforesaid compounds i.e. compounds of formula (I) (including the compounds of the provisos) and Examples 11 to 26 hereinafter referred to as “compounds of the invention”) are useful in therapy.

### DETAILED DESCRIPTION OF THE INVENTION

When carbocyclyl and heterocyclyl are substituted, they are typically substituted by 1 or 2 substituents (e.g. 1 substituent). Typically the substituent is methyl. More typically carbocyclyl and heterocyclyl groups are unsubstituted.

When aryl and heteroaryl are substituted, they are typically substituted by 1, 2 or 3 (e.g. 1 or 2) substituents. Substituents for aryl and heteroaryl are selected from C₁₋₆alkyl (e.g. methyl), C₂₋₆alkenyl (e.g. buten-3-yl), C₂₋₆alkynyl (e.g. butyn-3-yl), C₁₋₆haloalkyl (e.g. fluoromethyl, trifluoromethyl), —C₁₋₆thioalkyl (e.g. —S-methyl), —SO₂C₁₋₄alkyl (e.g. —SO₂-methyl), C₁₋₆alkoxy- (e.g. methoxy, ethoxy), —O—C₃₋₈cycloalkyl (e.g. —O-cyclopentyl), C₃₋₈cycloalkyl (e.g. cyclopropyl, cyclohexyl), —SO₂C₃₋₈cycloalkyl (e.g. —SO₂cyclohexyl), C₃₋₆alkenyloxy- (e.g. —O-buten-2-yl), C₃₋₆alkynyloxy- (e.g. —O-buten-2-yl), —C(O)C₁₋₆alkyl (e.g. —C(O)ethyl), —C(O)OC₁₋₆alkyl (e.g. —C(O)O-methyl), C₁₋₆alkoxy-C₁₋₆alkyl- (e.g. methoxy-ethyl-), nitro, halogen (e.g. fluoro, chloro, bromo, iodo), cyano, hydroxyl, —C(O)OH, —NH₂, —NHC₁₋₄alkyl (e.g. —NHmethyl), —N(C₁₋₄alkyl)(C₁₋₄alkyl) (e.g. —N(methyl)₂), —C(O)N(C₁₋₄alkyl)(C₁₋₄alkyl) (e.g. —C(O)N(methyl)₂), —C(O)NH₂ and —C(O)NH(C₁₋₄alkyl) (e.g. —C(O)NHmethyl). More typically, substituents will be selected from C₁₋₆alkyl (e.g. methyl), C₁₋₆haloalkyl (e.g. C₁₋₆fluoroalkyl, e.g. CF₃), C₁₋₆alkoxy (e.g. OMe), halogen and hydroxyl.

When W represents —C₁₋₆alkyl-aryl in which aryl is optionally substituted, examples include —C₁₋₆alkyl-phenyl and —C₁₋₆alkyl-naphthyl, (especially —C₁₋₆alkyl-phenyl), e.g. -methyl-phenyl, -ethyl-phenyl, -propyl-phenyl, -butyl-phenyl, -methyl-naphthyl and -ethyl-naphthyl wherein aryl is optionally substituted.

When W represents —C₂₋₆alkenylaryl, in which aryl is optionally substituted, examples include -(E)CH═CHPh and -(E)CH₂CH═CHPh.

When W represents —C₁₋₆alkylheteroaryl in which heteroaryl is optionally substituted, examples include —C₁₋₄alkylheteroaryl such as -methyl-pyridine, -methyl-furan, -methyl-thiophene, -methyl-pyrrole, -methyl(imidazole), -methyl(methylfuran).

When W represents —C₂₋₆alkenylheteroaryl in which heteroaryl is optionally substituted, examples include -(E)CH═CH-(pyrrolyl), -(E)CH₂CH═CH(pyrrolyl), -(E)CH═CH-(furanyl), -(E)CH₂CH═CH(furanyl), -(E)CH═CH-(thiphenyl) and -(E)CH₂CH═CH(thiophenyl).

When Y represents the side moiety of an analogue of an amino acid which is substituted, examples include

the side chain of an analogue of Phe in which the aromatic moiety is substituted by one more groups selected from halogen (e.g. fluoro, chloro, bromo, iodo), hydroxyl, nitro, C₁₋₄-alkyl (e.g. methyl or ethyl, especially methyl), C₁₋₄haloalkyl (e.g. fluoromethyl or trifluoromethyl), hydroxyl, C₁₋₄alkoxy (e.g. methoxy or ethoxy, especially methoxy) and C₁₋₄haloalkoxy (e.g. trifluoromethoxy), for example the aromatic moiety is represented by 2-fluorophenyl-, 4-fluorophenyl, 2-chlorophenyl-, 4-chlorophenyl-, 2-bromophenyl-, 4-bromophenyl-, 2-iodophenyl-, 4-iodophenyl-, 4-hydroxyphenyl- (i.e. the side chain of Tyr), 4-nitrophenyl-, 2-methylphenyl-, 3-methylphenyl-, 4-methylphenyl-, 2,4-dimethylphenyl-, 3,5-dimethylphenyl-, 4-trifluoromethylphenyl-, 4-methoxyphenyl- (i.e. the side chain of Tyr wherein the hydroxyl group has been substituted by methyl), 4-ethoxyphenyl- (i.e. the side chain of Tyr wherein the hydroxyl group has been substituted by ethyl), 2,4-dimethoxyphenyl- or 4-trifluoromethoxyphenyl-; or

the side chain of an analogue of Ser or Thr in which the hydroxyl group is substituted by methyl, ethyl or propyl (e.g. methyl); or

the side chain of an analogue of Trp in which the heteroaromatic moiety is substituted by one or more methyl groups, e.g. (1-methyl)indol-3-yl-, (2-methyl)indol-3-yl-, (4-methyl)indol-3-yl-,(5-methyl)indol-3-yl-, (6-methyl)indol-3-yl-, (7-methyl)indol-3-yl-, (5,7-dimethyl)indol-3-yl-; or

the side chain of an analogue of Cys in which the thiol group is substituted by methyl, ethyl or propyl (e.g. methyl); or

the side chain of an analogue of Asp or Glu wherein the carboxylic acid group has been converted into a methyl, ethyl or propyl ester (e.g. a methyl ester); or

the side chain of an analogue of Asn or Gln wherein the —NH₂ of the amide has been converted into an —NH(methyl), —NH(ethyl), —N(methyl)(methyl)-N(methyl)(ethyl) or —N(ethyl)(ethyl) group; or

the side chain of an analogue of Lys or Arg wherein the —NH₂ of the amine has been converted into an —NHC(O)methyl, —NHC(O)ethyl group, —N(methyl)C(O)methyl, —N(methyl)C(O)ethyl or —N(ethyl)C(O)methyl group.

When Z represents optionally substituted heteroaryl examples include furan-2-yl, furan-3-yl, pyrrol-2-yl, pyrrol-3-yl, thiophen-2-yl, thiophen-3-yl, pyridin-2-yl, pyridin-3-yl, pyridin-4-yl, benzofuran-2-yl, benzothiophen-2-yl, benzothiazol-2-yl, indol-2-yl, thiazol-2-yl, imidazol-2-yl, imidazol-2-yl.

When Z represents optionally substituted aryl, examples include optionally substituted phenyl and optionally substituted naphthyl.

When Z represents optionally substituted phenyl examples include optionally substituted phenyl and optionally substituted naphthyl, (especially optionally substituted phenyl) e.g. unsubstituted phenyl, 4-methylphenyl-, 2,4-dimethylphenyl-, 3,4,5-trimethylphenyl-, 2,4,6-trimethylphenyl-, 2-methoxyphenyl-, 3-methoxyphenyl-, 4-methoxyphenyl-, 2,4-dimethoxyphenyl-, 3,5-dimethoxyphenyl-, 2-trimethoxyphenyl-, 3-trimethoxyphenyl-, 4-trimethoxyphenyl-, 2,4-bis(trimethoxy)phenyl-, 3,5-bis(trimethoxy)phenyl-, 4-ethoxyphenyl-, 2-bromo-5-chlorophenyl-, 2-fluorophenyl-, 2-chlorophenyl-, 2-bromophenyl-, 3-fluorophenyl-, 3-chlorophenyl-, 3-bromophenyl-, 4-fluorophenyl-, 4-chlorophenyl- and 4-bromophenyl-. When Z represents optionally substituted naphthyl, examples include naphthyl, 3-methyl-naphthyl-, and 6-methyl-naphthyl-.

When W represents —C₁₋₆alkyl-aryl in which aryl is optionally substituted, W suitably represents benzyl wherein the phenyl ring is optionally substituted. Most suitably W represents unsubstituted benzyl.

When W represents —C₂₋₆alkenyl-aryl in which aryl is optionally substituted, W suitably represents -(E)CH═CH-Ph.

When X and Y are not joined, X suitably represents H.

When Z represents optionally substituted heteroaryl Z suitably represents benzothiophen-2-yl, benzothiazol-2-yl, furan-2-yl, pyridin-2-yl, thiazol-2-yl or thiophen-2-yl.

When Z represents optionally substituted phenyl Z suitably represents unsubstituted phenyl.

Suitably K represents O.

Suitably W represents —C₁₋₄alkyl-aryl (e.g. in which aryl represents optionally substituted phenyl). Most suitably, W represents benzyl.

Suitably X represents H and Y represents the side chain of Ala, Leu, Trp or Phe or the side chain of an analogue of Phe in which the aromatic moiety is substituted; or X and Y are joined such that

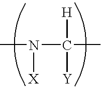

represents

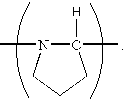

Most suitably X represents H and Y represents the side chain of Ala, Leu, Trp or Phe or the side moiety of an analogue of Phe in which the aromatic moiety is represented by 4-iodophenyl or 4-nitrophenyl and particularly represents the side chain of Phe.

Alternatively, suitably X and Y are joined such that

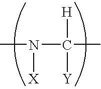

represents

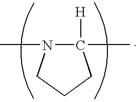

In one embodiment of the invention Z represents heteroaryl which may optionally be substituted. In another embodiment of the invention Z represents aryl which may optionally be substituted.

When Z represents heteroaryl, suitably Z represents pyridinyl or a five membered heteroaryl group containing one or two heteroatoms optionally fused to a phenyl ring wherein any of the aforesaid pyridinyl, heteroaryl or phenyl may optionally be substituted (e.g. by methyl). More suitably Z represents a five membered heteroaryl group containing one or two heteroatoms optionally fused to a phenyl ring. Most suitably Z is unsubstituted. Most suitably Z represents benzothiophen-2-yl, benzthiazol-2-yl, furan-2-yl, pyridin-2-yl, thiazol-2-yl or thiophen-2-yl.

When Z represents aryl, suitably Z represents phenyl, which may optionally be substituted (e.g. by methyl). Most suitably Z is unsubstituted.

Suitably the stereochemistry at * is the same as that of the naturally occurring L-amino acid or analogue thereof.

The PEP-inhibitors of the present invention are shown to be effective to modulate the basal level of interleukin-6 (IL-6) in human glial cells. These compounds show a significant suppression of IL-6 secretion.

IL-6, a pleiotropic cytokine, contributes to a multitude of neuropathological and pathophysiological processes, especially in inflammation, cancer, infection and autoimmune diseases. Overexpression of IL-6 has been implicated in the pathology of multiple myeloma, solid tumors, prostatic cancers, bladder cancers, neurological cancers, Castleman's disease, inflammation, myocardial infarction, Paget's disease, ischemia, asthma, rheumatoid arthritis, psoriasis, Alzheimer's disease, multiple sclerosis, meningitis, stroke, osteoporosis, insulin resistance, obesity, impaired glucose tolerance, type 2 diabetes, cancer-related anorexia and cachexia as well as multidrug resistance. Therefore, reduction of pathological IL-6 concentrations by compounds which are described here may be useful in treatment of IL-6 related diseases, for instance those mentioned above.

Furthermore, the PEP-inhibitors of the present invention are shown to be surprisingly effective at modulating the basal level of beta-amyloid peptides, especially of Abeta₁₋₄₀ and Abeta₁₋₄₂ in different human cell lines, e.g. neuronal cells. The compounds of the present invention show a significant increase of the secretion of beta-amyloid peptides.

Beta-amyloid peptides are considered to be the cause of neurodegeneration and neuronal cell death in patients faced with MCI (Mild Cognitive Impairment) Alzheimer's disease (AD) and for the progression of MCI to AD. Recently, it was shown that the β-amyloid species, which are involved in the onset of MCI and AD, are formed intracellularly. Moreover, not the full-length peptides Abeta₁₋₄₀ and Abeta₁₋₄₂ but N-terminally truncated and N-terminally modified forms of beta-amyloid peptides, e.g. Abeta₃₋₄₀, Abeta₃₋₄₂, pGlu-Abeta₃₋₄₀, pGlu-Abeta₃₋₄₂, Abeta₁₁₋₄₂ and pGlu-Abeta1₁₋₄₂ are discussed as the toxic forms (36).

The compounds of the present invention should therefore be useful to prevent the formation of neurotoxic β-amyloid peptides, e.g. Abeta₃₋₄₀, Abeta₃₋₄₂, pGlu-Abeta₃₋₄₀, pGlu-Abeta₃₋₄₂, Abeta₁₁₋₄₂ and pGlu-Abeta₁₁₋₄₂ by enhancement of the secretion of full-length Abeta₁₋₄₀ and Abeta₁₋₄₂ before N-terminal truncation and modification.

Furthermore, it was recently demonstrated that the peptide humanin is a substrate for PEP. Specifically, it was demonstrated that prolyl endopeptidase is able to cleave the peptide humanin at two positions in the peptide sequence, after the proline residue in position 3 and after the cysteine residue at position 8. This cleavage pattern can be completely inhibited by the use of specific PEP inhibitors.

Humanin was originally discovered by means of a unbiased functional screening for genes suppressing FAD (familial Alzheimer's disease) and Abeta induced neuronal cell death, respectively (30;32). The peptide is an unusually 75 by gene product of the mitochondrial 16S ribosomal RNA (27;30). The evidence for a cellular expression of this gene product was given by Western blots using a peptide-antibody (33). A detailed analysis of the physiological activity revealed the existence of a humanin core domain [residues 3 to 19] (34;35). In particular conservation of seven residues like Pro[3], Cys[8], Leu[9], Leu[12], Thr[13], Ser[14] and Pro[19] turned out to be essential (34;35). Replacement of these residues by alanine or abbreviation of the core sequence results in a loss of the apoptosis rescue ability of humanin.

Humanin was recently highlighted for its ability to suppress apoptosis by interacting with the Bcl2-associated X protein (Bax) (27). An additional interaction with the insulin-like growth factor binding protein-3 (IGFBP-3), thereby blocking the IGFBP-3 induced cell death in glioblastoma cells, supports humanin's cell survival promoting capacity (28). The 24 amino acid peptide is able to preserve cortical neurons from prion-peptide- or amyloid-β induced insults (29), improves impaired metabolic activity and prolongs survival of serum-deprived human lymphocytes (31).

Accordingly, PEP-inhibitors are useful for the prevention of the degradation of peptide substrates, which can be degraded by post cysteine cleavage, e.g. the peptide humanin. Furthermore, the present invention provides a method for the prevention of the degradation of peptide substrates, which can be degraded by post cysteine cleavage, e.g. the peptide humanin. The compounds of the invention are especially suitable for use in this method.

The compounds of the present invention have several unique and surprising properties and are expected to be useful for the treatment of neurodegenerative diseases, e.g. MCI, AD, Down Syndrome, Parkinson disease and Chorea Huntington.

The present invention provides the compounds of the invention for use as a medicament. The compounds of the invention are inhibitors of PEP and PEP-like enzymes.

Furthermore, the present invention provides the use of inhibitors of PEP and PEP-like enzymes of the invention for the preparation of a medicament for the treatment of a disease selected from the group consisting of Alzheimer's disease, Down Syndrome, Parkinson disease, Chorea Huntington, pathogenic psychotic conditions, schizophrenia, impaired food intake, sleep-wakefulness, impaired homeostatic regulation of energy metabolism, impaired autonomic function, impaired hormonal balance, impaired regulation, body fluids, hypertension, fever, sleep dysregulation, anorexia, anxiety related disorders including depression, seizures including epilepsy, drug withdrawal and alcoholism, neurodegenerative disorders including cognitive dysfunction, dementia, aphasia, apraxia, agnosia, or any type of amnesias, mild cognitive impairment (MCI), benign forgetfulness and Korsakoff's syndrome, pulmonary vascular disease, restenosis or pulmonary hypertension myocarditis, bronchopulmonary dysplasia, myocardial necrosis or post-cardiac transplant coronary arteriopathy, atherosclerosis, reperfusion injury, hypoxia, ischemia and blood coagulation disorders.

The present invention also provides inhibitors of PEP and PEP-like enzymes of the invention for use in the treatment of a disease selected from the group consisting of Alzheimer's disease, Down Syndrome, Parkinson disease, Chorea Huntington, pathogenic psychotic conditions, schizophrenia, impaired food intake, sleep-wakefulness, impaired homeostatic regulation of energy metabolism, impaired autonomic function, impaired hormonal balance, impaired regulation, body fluids, hypertension, fever, sleep dysregulation, anorexia, anxiety related disorders including depression, seizures including epilepsy, drug withdrawal and alcoholism, neurodegenerative disorders including cognitive dysfunction, dementia, aphasia, apraxia, agnosia, or any type of amnesias, mild cognitive impairment (MCI), benign forgetfulness and Korsakoff's syndrome, pulmonary vascular disease, restenosis or pulmonary hypertension myocarditis, bronchopulmonary dysplasia, myocardial necrosis or post-cardiac transplant coronary arteriopathy, atherosclerosis, reperfusion injury, hypoxia, ischemia and blood coagulation disorders.

The present invention also provides a method of treatment for a disease selected from the group consisting of Alzheimer's disease, Down Syndrome, Parkinson disease, Chorea Huntington, pathogenic psychotic conditions, schizophrenia, impaired food intake, sleep-wakefulness, impaired homeostatic regulation of energy metabolism, impaired autonomic function, impaired hormonal balance, impaired regulation, body fluids, hypertension, fever, sleep dysregulation, anorexia, anxiety related disorders including depression, seizures including epilepsy, drug withdrawal and alcoholism, neurodegenerative disorders including cognitive dysfunction, dementia, aphasia, apraxia, agnosia, or any type of amnesias, mild cognitive impairment (MCI), benign forgetfulness and Korsakoff's syndrome, pulmonary vascular disease, restenosis or pulmonary hypertension myocarditis, bronchopulmonary dysplasia, myocardial necrosis or post-cardiac transplant coronary arteriopathy, atherosclerosis, reperfusion injury, hypoxia, ischemia and blood coagulation disorders, comprising the administration of a therapeutically active amount of at least one compound of the invention to a mammal, preferably a human.

Most preferably, the present invention provides a method of treatment and corresponding uses for a disease selected from the group consisting of mild cognitive impairment (MCI), Alzheimer's disease, Down Syndrome, Parkinson disease and Chorea Huntington, comprising the administration of a therapeutically active amount of at least one compound of the invention to a mammal, preferably a human.

In a further embodiment, the compounds of the invention are useful to inhibit microbial growth, reduce perioperative blood loss, preserve transplantation tissues or organs, inhibit cancer cell growth or tumor progression or tumor metastasis or invasion.

Combinations

In a further embodiment, the present invention provides a composition, preferably a pharmaceutical composition comprising at least one compound of the invention optionally in combination with at least one compound selected from the group consisting of inhibitors of glutaminyl cyclase (QC), LiCl, inhibitors of dipeptidyl aminopeptidases, preferably inhibitors of DP IV or DP IV-like enzymes, NPY-receptor ligands, NPY agonists, acetylcholinesterase (AChE) inhibitors, protein isoaspartate carboxymethyl transferase (PIMT) enhancers, inhibitors of beta secretases, inhibitors of gamma secretases, inhibitors of neutral endopeptidase, inhibitors of Phosphodiesterase-4 (PDE-4), monoamine oxidase (MAO) inhibitors, TNFalpha inhibitors, amyloid protein or amyloid peptide deposition inhibitors, sigma-1 receptor inhibitors and histamine H3 antagonists.

These combinations provide a particularly beneficial effect on behavioral conditions and such combinations are therefore shown to be effective and useful for the treatment of a disease selected from the group consisting of Alzheimer's disease, Down Syndrome, Parkinson disease, Chorea Huntington, pathogenic psychotic conditions, schizophrenia, impaired food intake, sleep-wakefulness, impaired homeostatic regulation of energy metabolism, impaired autonomic function, impaired hormonal balance, impaired regulation, body fluids, hypertension, fever, sleep dysregulation, anorexia, anxiety related disorders including depression, seizures including epilepsy, drug withdrawal and alcoholism, neurodegenerative disorders including cognitive dysfunction, dementia, aphasia, apraxia, agnosia, or any type of amnesias, mild cognitive impairment (MCI), benign forgetfulness and Korsakoff's syndrome, pulmonary vascular disease, restenosis or pulmonary hypertension myocarditis, bronchopulmonary dysplasia, myocardial necrosis or post-cardiac transplant coronary arteriopathy, atherosclerosis, reperfusion injury, hypoxia, ischemia and blood coagulation disorders.

The combinations of the present invention are further useful to inhibit microbial growth, reduce perioperative blood loss, preserve transplantation tissues or organs, inhibit cancer cell growth or tumor progression or tumor metastasis or invasion.

Pharmaceutical Compositions

The invention provides pharmaceutical compositions containing at least one compound of the invention optionally in combination with at least one agent as mentioned for the combinations above, together with one or more therapeutically acceptable diluents or carriers. The active ingredient(s) is intimately admixed with a pharmaceutical diluent or carrier according to conventional pharmaceutical compounding techniques, which diluent or carrier may take a wide variety of forms depending of the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenteral administration, the carrier will usually comprise sterile water, though other ingredients, for example, for purposes such as aiding solubility or for preservation, may be included.

Soluble polymers as targetable drug carriers can include polyvinylpyrrolidone, pyran copolymer, polyhydroxypropylmethacrylamidephenol, polyhydroxyethylaspartamidephenol, or polyethyleneoxidepolyllysine substituted with palmitoyl residue. Furthermore, the compounds of the present invention may be coupled to a class of biodegradable polymers useful in achieving controlled release of a drug, for example, polyactic acid, polyepsilon caprolactone, polyhydroxy butyeric acid, polyorthoesters, polyacetals, polydihydropyrans, polycyanoacrylates and cross-linked or amphipathic block copolymers of hydrogels.

Suitable binders include, without limitation, starch, gelatin, natural sugars such as glucose or betalactose, corn sweeteners, natural and synthetic gums such as acacia, tragacanth or sodium oleate, sodium stearate, magnesium stearate, sodium benzoate, sodium acetate, sodium chloride and the like.

Disintegrators include, without limitation, starch, methyl cellulose, agar, bentonite, xanthan gum and the like.

Injectable suspensions may also prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient(s) necessary to deliver an effective dose as described above. The pharmaceutical compositions herein will contain, per dosage unit, e.g., tablet, capsule, powder, injection, suppository, teaspoonful and the like, from about 0.03 mg to 100 mg/kg (preferred 0.1-30 mg/kg) and may be given at a dosage of from about 0.1-300 mg/kg per day (preferred 1-50 mg/kg per day) of each active ingredient or combination thereof. The dosages, however, may be varied depending upon the requirement of the patients, the severity of the condition being treated and the compound being employed. The use of either daily administration or post-periodic dosing may be employed.

Preferably these compositions are in unit dosage forms from such as tablets, pills, capsules, powders, granules, sterile parenteral solutions or suspensions, metered aerosol or liquid sprays, drops, ampules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid preformulation composition containing a homogeneous mixture of a compound of the present invention, or a pharmaceutically acceptable salt thereof. When referring to these preformulation compositions as homogeneous, it is meant that the active ingredient is dispersed evenly throughout the composition so that the composition may be readily subdivided into equally effective dosage forms such as tablets, pills and capsules. This solid preformulation composition is then subdivided into unit dosage forms of the type described above containing from 0.1 to about 500 mg of each active ingredient or combinations thereof of the present invention.

The tablets or pills of the compositions of the present invention can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

This liquid forms in which the compositions of the present invention may be incorporated for administration orally or by injection include, aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinylpyrrolidone or gelatin.

Where the processes for the preparation of the compounds of the present invention give rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their components enantiomers by standard techniques, such as the formation of diastereomeric pairs by salt formation with an optically active acid, such as (−)-di-p-toluoyl-d-tartaric acid and/or (+)-di-p-toluoyl-l-tartaric acid followed by fractional crystallization and regeneration of the free base. The compounds may also resolved by formation of diastereomeric esters or amides, followed by chromatographic separation and removal of the chiral auxiliary. Alternatively, the compounds may be resolved using a chiral HPLC column.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or betalactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms in suitable flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

The compounds or combinations of the present invention can also be administered in the form of liposome delivery systems, such as small unilamellar vesicles, large unilamellar vesicles, and multilamellar vesicles. Liposomes can be formed from a variety of phospholipids, such as cholesterol, stearylamine or phosphatidylcholines.

Compounds or combinations of the present invention may also be delivered by the use of monoclonal antibodies as individual carriers to which the compound molecules are coupled. The compounds of the present invention may also be coupled with soluble polymers as targetable drug carriers. Such polymers can include polyvinylpyrrolidone, pyran copolymer, polyhydroxypropylmethacrylamidephenol, polyhydroxyethylaspartamid-ephenol, or polyethyl eneoxidepolyllysine substituted with palmitoyl residue. Furthermore, the compounds of the present invention may be coupled to a class of biodegradable polymers useful in achieving controlled release of a drug, for example, polyactic acid, polyepsilon caprolactone, polyhydroxy butyeric acid, polyorthoesters, polyacetals, polydihydropyrans, polycyanoacrylates and cross-linked or amphipathic block copolymers of hydrogels.

Compounds or combinations of this invention may be administered in any of the foregoing compositions and according to dosage regimens established in the art whenever treatment of the addressed disorders is required.

The daily dosage of the products may be varied over a wide range from 0.01 to 1.000 mg per mammal per day. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250 and 500 milligrams of each active ingredient or combinations thereof for the symptomatic adjustment of the dosage to the patient to be treated. An effective amount of the drug is ordinarily supplied at a dosage level of from about 0.1 mg/kg to about 300 mg/kg of body weight per day. Preferably, the range is from about 1 to about 50 mg/kg of body weight per day. The compounds or combinations may be administered on a regimen of 1 to 4 times per day.

Optimal dosages to be administered may be readily determined by those skilled in the art, and will vary with the particular compound used, the mode of administration, the strength of the preparation, the mode of administration, and the advancement of disease condition. In addition, factors associated with the particular patient being treated, including patient age, weight, diet and time of administration, will result in the need to adjust dosages.

Suitably, in the case of combinations according to the invention, the particularly beneficial effect provided by the treatment of the invention is an improved therapeutic ratio for the combination of the invention relative to the therapeutic ratio for one compound of the combination when used alone and at a dose providing an equivalent efficacy to the combination of the invention.

In a preferred aspect, the particularly beneficial effect provided by the treatment of the invention is indicated to be a synergistic effect relative to the control expected from the effects of the individual active agents.

In a further aspect of the invention, combining doses of at least one compound of the invention with at least one agent as defined for the combinations herein will preferably produce a greater beneficial effect than can be achieved for either agent alone at a dose twice that used for that agent in the combination.

In a preferred aspect, the dosage level of each of the active agents when used in accordance with the treatment of the invention will be less than would have been required from a purely additive effect upon the neuronal condition.

Without being limited by theory, it is also considered that the treatment of the invention may effect an improvement, relative to the individual agents, in decreasing the intracellular deposition of pGlu-amyloid-beta-peptides and thereby dramatically slowing down the plaque formation in the brain of a mammal, preferably in human brain.

In a further aspect, the invention also provides a process for preparing a pharmaceutical composition comprising at least one at least one compound of the invention optionally in combination with at least one agent as defined for the combinations herein and a pharmaceutically acceptable carrier therefore, which process comprises admixing the compound of the invention and said optional agent(s) and a pharmaceutically acceptable diluent or carrier.

The compositions are preferably in a unit dosage form in an amount appropriate for the relevant daily dosage.

Suitable dosages, including especially unit dosages, of the compounds of the invention, QC-inhibitors, LiCl, inhibitors of dipeptidyl aminopeptidases, preferably inhibitors of DP IV or DP IV-like enzymes, NPY-receptor ligands, NPY agonists, acetylcholinesterase (AChE) inhibitors, PIMT enhancers, inhibitors of beta secretases, inhibitors of gamma secretases, inhibitors of neutral endopeptidase, inhibitors of Phosphodiesterase-4 (PDE-4), monoamine oxidase (MAO) inhibitors, TNFalpha inhibitors, amyloid protein or amyloid peptide deposition inhibitors, sigma-1 receptor inhibitors and histamine H3 antagonists include the known dosages including unit doses for these compounds as described or referred to in reference text such as the British and US Pharmacopoeias, Remington's Pharmaceutical Sciences (Mack Publishing Co.), Martindale The Extra Pharmacopoeia (London, The Pharmaceutical Press) (for example see the 31st Edition page 341 and pages cited therein) or the above mentioned publications.

Preferred compounds of the invention are those having an IC₅₀ value or a Kᵢ value, and preferably an IC₅₀ value and a K value, of less than 1×10⁻⁶, in particular less than 1×10⁻⁷ and especially less than 1×10⁻⁸ M.

Preferred compounds of the invention have a molecular weight of less than 2000 Da especially less than 1000 Da particularly less than 600 Da, e.g. less than 500 Da.

Compounds and combinations of the invention may have the advantage that they are, for example, more potent, more selective, have fewer side-effects, have better formulation and stability properties, have better pharmacokinetic properties, be more bioavailable, be able to cross blood brain barrier and are more effective in the brain of mammals, are more compatible or effective in combination with other drugs or be more readily synthesized than other compounds of the prior art.

The invention embraces all combinations of preferred and more preferred groups and embodiments of groups recited above.

### EXAMPLES

### Biological Evaluation, Determination of IC₅₀- and Kᵢ-Values of PEP-Inhibitors

Recombinant human prolyl oligopeptidase was used for measurement. Recombinant expression was performed in E. coli under standard conditions as described elsewhere in the state of the art.

For activity measurements the chromogenic substrate Cbz-Gly-L-Pro-pNA was used in HEPES buffer pH 7.6 containing 50 mM HEPES, 200 mM NaCl, 1 mM EDTA, 1 mM DTT, 0.006% Brij35. Measurements were carried out at 30° C. Release of pNA were monitored continuously at 405 nm.

IC₅₀ values were determined using one substrate concentration (0.15 mM) and 11-15 serial dilutions of inhibitor starting with 0.1 mM. IC₅₀ values were calculated using non-linear regression to a 4-parameter equation (Prism 4.0, GraphPad).

For Kᵢ determination 4 substrate (0.15 mM, 0.08 mM 0.04 mM, 0.02 mM) and 7 inhibitor concentrations in an appropriate range were used. Calculations were performed by multiple non-linear regression analysis to the equation for competitive inhibition using GraFit 5.0 Software (Erithacus Software).

IL-6 ELISA

To analyze basal secretion of IL-6, human glial U-343 cells were cultured in 6 well plates (1.5×10⁶ cells/well, Greiner) and treated with specific PEP inhibitors as indicated (20 μM each) for 24 hours in serum-free D-MEM medium (Invitrogen). Aliquots of 40 μl conditioned medium were used to quantify the amount of secreted IL-6 by an human-specific IL-6 ELISA (Biosource) following the manufacturer's instructions. All data were obtained in quadruplicate. For the calculation of the IL-6 concentration in the cell culture medium after PEP-inhibitor treatment, the basal IL-6 concentration of the cell culture medium of untreated cell samples was set to 100%. The results of the measurement of the IL-6 concentration with PEP-inhibitor treated cells are presented as % of the untreated cell samples.

Cell Culture

The human glioma cell line, U-343 and the human neuroblastoma cell line, SH-SY5Y were maintained in Dulbecco's modified Eagle's medium (DMEM) containing 10% fetal bovine serum (Gibco BRL, Karlsruhe, Germany) and incubated at 37° C. in a 5% CO₂ atmosphere. Culture media contained in general 60 μg/ml gentamycin (Gibco BRL, Karlsruhe, Germany).

Beta-Amyloid ELISA

To quantify intracellular and extracellular concentrations of beta-amyloid peptides, 1-40 and 1-42, U-343 and SH-SY5Y cells were cultured in 6 well plates (1.5×10⁶ cells/well) and treated with specific PEP inhibitors (20 μM each) for 24 hours. For quantitation of secreted beta-amyloid peptides the conditioned medium was collected and concentrated by lyophylisation. Likewise, after determination of the cell numbers/well (casy cell counter I, Schärfe System, Reutlingen, Germany), cells were lyzed with cell extraction buffer (Biosource, Solingen, Germany) according to the manufacturer's protocol. The protein concentration was determined by the method of Bradford (1976). Aliquots of 100 μl were used to quantify beta-amyloid peptides 1-40 and 1-42 in quadruplicate by ELISA (IBL, Hamburg, Germany) following the manufacturer's instructions. All obtained intracellular and extracellular concentrations were normalized to cell numbers and protein concentration, respectively.

For the calculation of the concentration of beta-amyloid peptides 1-40 and 1-42 in the cell culture medium after PEP-inhibitor treatment, the basal concentration of beta-amyloid peptides 1-40 and 1-42 in the cell culture medium of untreated cell samples was set to 100%. The results of the measurement of the concentration of beta-amyloid peptides 1-40 and 1-42 with PEP-inhibitor treated cells are presented as % of the untreated cell samples.

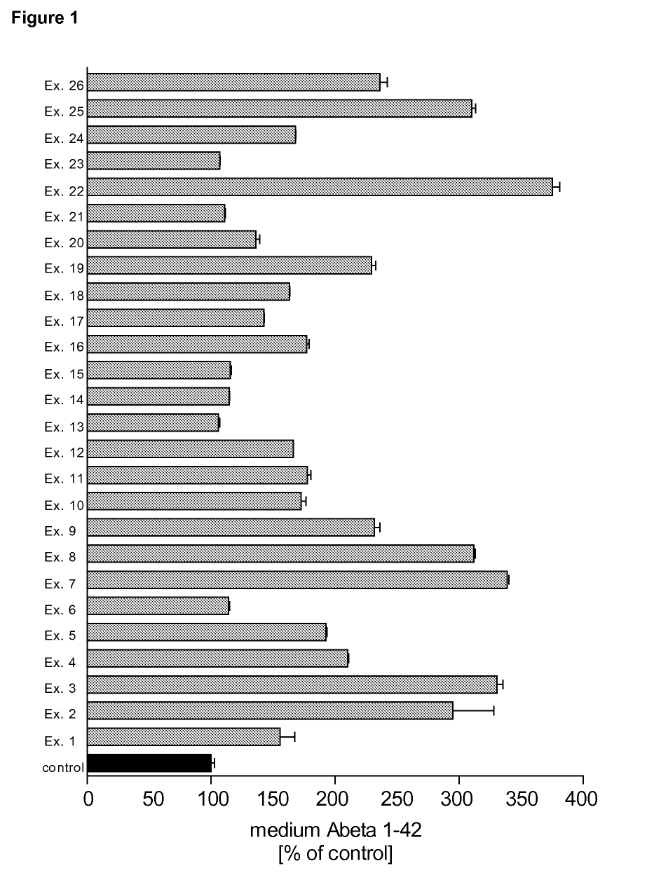

The conditioned medium of human glial U343 cells, treated with PEP inhibitors over 24 hours contained only 18% to 60% of IL-6 amount measured in untreated control samples (FIG. 1). Values are presented as mean±SD of quadruplicate wells and were analyzed for statistical significance by unpaired t test (***p<0.001).

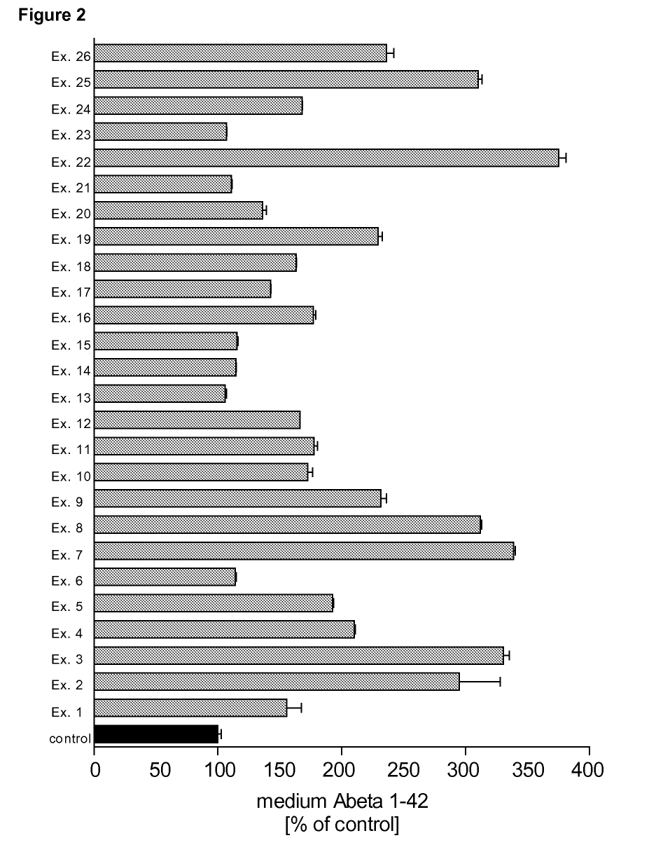

The conditioned medium of human glial U343 cells, treated with PEP inhibitors over 24 hours contained 87.5% to 546% of Abeta 1-42 amount measured in untreated control samples (FIG. 2). Values are presented as mean±SD of quadruplicate wells and were analyzed for statistical significance by unpaired t test (***p<0.001).

Thus, PEP inhibitors of the present invention show a significant reduction of the IL-6 level and an increased beta-amyloid secretion, especially of beta-amyloid peptides 1-42.

Example Compounds

| | | | | | | Extracellu- | |
|---|---|---|---|---|---|---|---|
| | | | | | | lar IL-6 | Extracellu- |
| | | | | | | concen- | lar Abeta- |
| | | | | | HPLC^{a}, | tration^{b}, | concen- |
| | | | | | Retention | % of | tration |
| | Structure | ESI-MS | IC₅₀ | Kᵢ | Time | untreated | % of |
| Ex. | Mᵣ (g/mol) | (M + H⁺) | PEP (M) | PEP (M) | (min.) | cells | control |
| 1 | 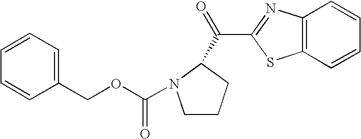 366.43 | 367.2 | 7.0 * 10⁻⁶ | 9.8 * 10⁻⁷ | 21.8 (A) | 40 | 155 |
| 2 | 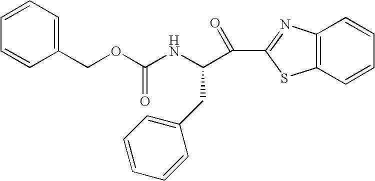 416.50 | 417.5 | >10⁻⁴ | n.d. | 23.0 (A) | 44 | 295 |
| 3 | 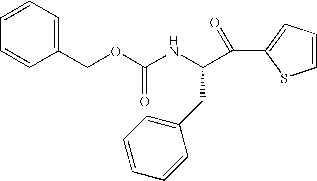 365.45 | 366.2 | 1.0 * 10⁻⁶ | n.d. | 21.4 (A) | 23 | 339 |
| 4 | 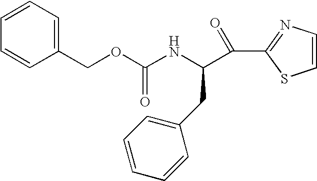 366.43 | 367.2 | n.i. | n.d. | 20.7 (A) | 44 | 172 |
| 5 | 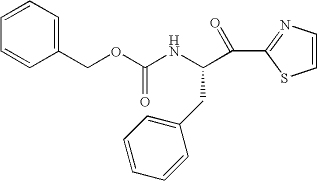 366.43 | 367.1 | 1.2 * 10⁻⁹ | 1.15 * 10⁻⁹ | 22.5 (B) | 29 | 193 |
| 6 | 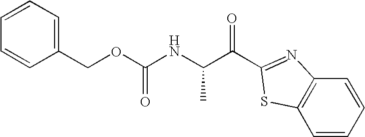 340.40 | 341.0 | 4.5 * 10⁻⁶ | n.d. | 18.6 (A) | 36 | 114 |
| 7 | 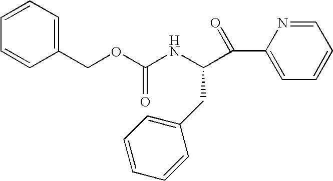 360.41 | 361.3 | 3.9 * 10⁻⁵ | n.d. | 21.0 (A) | 31 | 236 |
| 8 | 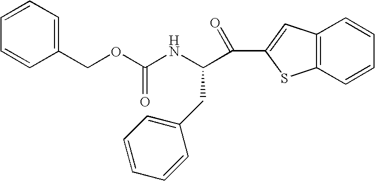 415.50 | 416.4 | n.i. | n.d. | 23.1 (A) | 27 | 310 |
| 9 | 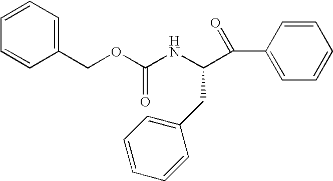 359.42 | 360.4 | 5.2 * 10⁻⁶ | 1.6 * 10⁻⁶ | 20.8 (A) | 18 | 330 |
| 10 | 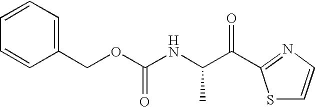 290.34 | 291.1 | 7.1 * 10⁻⁶ | 2.3 * 10⁻⁶ | 13.2 (A) | 78 | 107 |
| 11 | 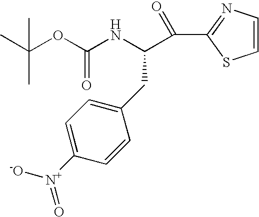 377.41 | 378.3 | 2.5 * 10⁻⁵ | n.d. | 17.6 (A) | 62 | 178 |
| 12 | 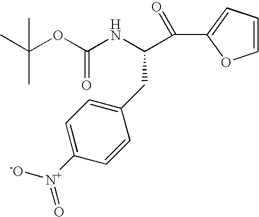 360.36 | 361.1 | 1.4 * 10⁻⁵ | n.d. | 18.6 (A) | 37 | 166 |
| 13 | 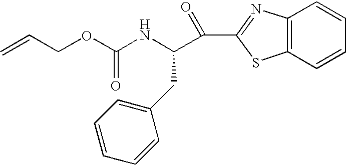 366.43 | 367.1 | n.i. | n.d. | 28.8 (A) | 69 | 106 |
| 14 | 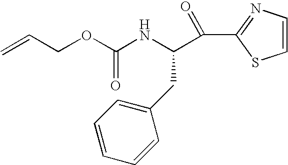 316.37 | 317.2 | 1.6 * 10⁻⁴ | n.d. | 15.8 (A) | 67 | 115 |
| 15 | 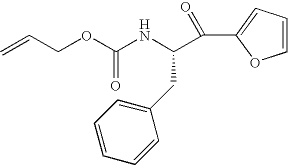 299.32 | 300.3 | 1.0 * 10⁻⁵ | n.d. | 14.4 (A) | 78 | 116 |
| 16 | 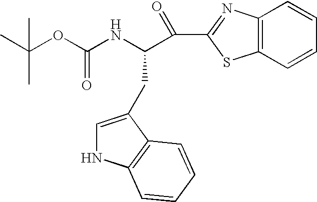 421.51 | 422.1 | 4.0 * 10⁻⁵ | n.d. | 21.8 (A) | 23 | 177 |
| 17 | 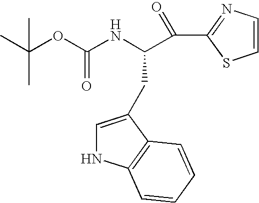 371.45 | 372.2 | 9.4 * 10⁻⁵ | n.d. | 17.2 (A) | 38 | 143 |
| 18 | 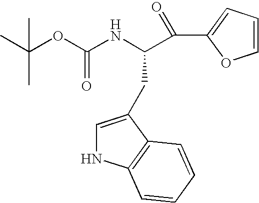 354.40 | 355.3 | 1.4 * 10⁻⁵ | n.d. | 16.0 (A) | 24 | 163 |
| 19 | 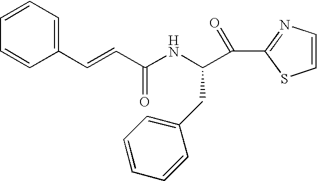 362.44 | 363.2 | n.i. | n.d. | 19.1 (A) | 58 | 230 |
| 20 | 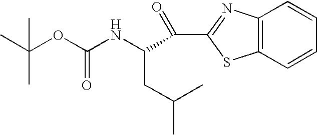 348.46 | 349.2 | 1.1 * 10⁻³ | n.d. | 25.0 (A) | 63 | 375 |
| 21 | 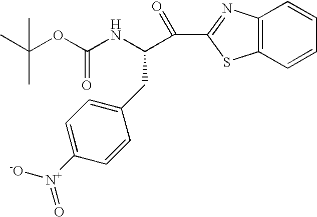 427.47 | 428.3 | n.i. | n.d. | 24.4 (A) | 96 | 311 |
| 22 | 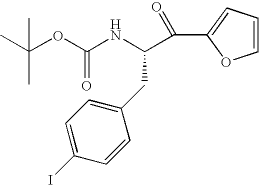 441.26 | 442.2 | n.i. | n.d. | 20.6 (A) | 50 | 232 |
| 23 | 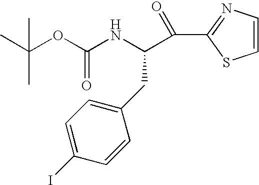 458.31 | 459.3 | n.i. | n.d. | 22.1 (A) | 41 | 210 |
| 24 | 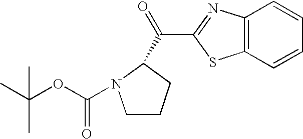 332.42 | 333.0 | 1.8 * 10⁻⁴ | n.d. | 19.9 (A) | 26 | 136 |
| 25 | 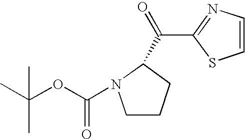 282.36 | 283.4 | 7.3 * 10⁻⁶ | 2.3 * 10⁻⁶ | 13.3 (A) | 59 | 111 |
| 26 | 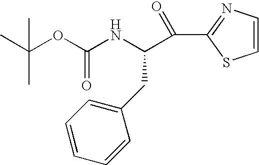 332.42 | 333.3 | 7.7 * 10⁻5 | 5.2 * 10⁻⁵ | 18.4 (A) | 66 | 168 |

^{a}Gradient A or B, described in the experimental section, the corresponding gradient is
mentioned in parenthesis.
^{b}Calculated with the untreated control sample as basal level,
n.d. means “not determined” ;
n.i. means “no inhibition”.

Processes

A process for preparation of a compound of formula (I)

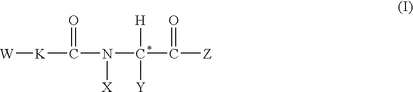

comprises reaction of a compound of formula (II)

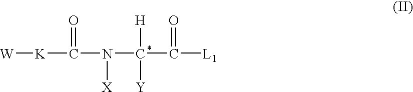

wherein W, K, X and Y are defined as above and L₁ represents a suitable leaving group [such as wherein —C(O)L₁ represents a Weinreb amide, i.e. —C(O)N(Me)(OMe)] with a compound of formula (III)

L₂-Z (III)

or a protected derivative thereof wherein Z is defined as above and L₂ represents an appropriate group (e.g. H or halogen e.g. Br) for the metallation.

The reaction may typically be carried out in the presence of an organometallic reagent, which acts as a metallation agent (e.g. n-butyllithium when Z represents heteroaryl or s-butyllithium when Z represents either heteroaryl or aryl).

Preparation of a Compound of Formula (II) Comprises Reaction of a Compound of Formula (IV)

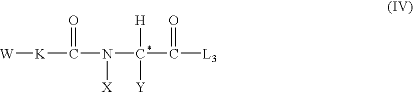

wherein L₃ represents a leaving group such as —OC(O)OMe or —OC(O)OEt or —OC(O)OCHMe₂

with a compound of formula (V) in the presence of a base.

H-L₁ (V)

e.g. a compound of formula (V) represents HN(Me)(OMe).

Compounds of formula (V) may be used in the form of a salt.

Compounds of formula (IV) need not be isolated before onward reaction to the compound of formula (II).

Preparation of a Compound of Formula (IV) Comprises Reaction of a Compound of Formula (VI)

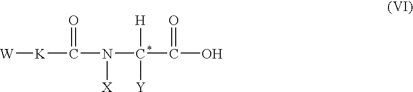

with a suitable reagent according to conventional methods known in the art. For example, when L₃ represents —OC(O)OMe, preparation of a compound of formula (IV) may comprise reaction of a compound of formula (VI) with an acid anhydride, e.g. MeOC(O)OC(O)OMe or an acid halogenide, e.g. ClC(O)OC(O)OMe.

Preparation of a Compound of Formula (VI) with K═O (Urethanes), K═S (Thiourethanes) and K═NH (Ureas) Comprises Reaction of a Compound of Formula (VII)

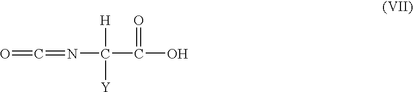

with a suitable counterpart according to conventional methods known in the art. Conversion to NX wherein X═H to NX wherein X=Me comprises an alkylation step according to conventional methods.

Alternatively, preparation of a compound of formula (VI) with K═O may comprise reaction of a compound of formula (VIII)

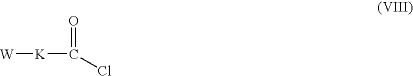

or an analogue in which chlorine is replaced by another halogen or similar leaving group with a suitable amine (IX)

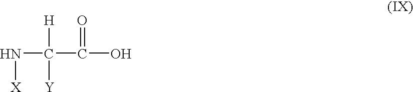

according to conventional methods known in the art.

Compounds of formulae (III), (VII), (VIII) and (IX) are either known or may be prepared by conventional methods known per se.

Analytical Methods

ESI-MS: Mass spectra were taken with an MDS Sciex API 365 mass spectrometer equipped with an Ionspray™ interface (MDS Sciex; Thorn Hill, ON, Canada). The instrument settings, data acquisition and processing were controlled by the Applied Biosystems (Foster City, Calif., USA) Analyst™ software for Windows NT™. 50-100 scans were performed by the positive ionization Q1 scan mode to accumulate the peaks. Sample solutions were diluted with 50% methanol in 0.5% formic acid to reach concentrations about 10 μg/ml. Each sample solution was introduced directly by a microsyringe (1 ml) through an infusion pump (Havard Apperatus 22; Havard Instruments; Holliston, Mass., USA) and fused silica capillary tubing at a rate of 20 ul/min. Thin layer chromatography (TLC) was done using Macherey Nagel Polygram® SIL G/UV₂₄₅. Visualisation was accomplished by means of UV light at 254 nm, followed by dyeing with potassium permanganate or Cer-Molybdate-solution. Solvents were distilled prior to use. All commercially available reagents were used without further purification. The amino acid derivates were purchased from Bachem. The pH-7 buffer solution used in the workup procedures was prepared by dissolving potassium dihydrogen phosphate (85.0 g) and sodium hydroxide (14.5 g) in water (1 l). Analytical HPLC was performed using a Merck-Hitachi device: acetonitrile-water (flow rate: 1 ml min⁻¹), column: LiChrosphere 5 um RP18e, 125×4.0 mm (Merck), pump: L-7100 Merck-Hitachi was used. Gradient A was used for the detection of the purified compounds in the examples. Characterisation of gradient A: starting from acetonitrile-water (20/80) at t=0 min to acetonitrile-water (95/5) within 30 min. Characterisation of gradient B: starting from acetonitrile-water (5/95) at t=0 min to acetonitrile-water (60/40) within 20 min, to acetonitrile-water (95/5) after additional 10 min.

General Synthetic Methods for the Preparation of the Starting Materials and the Examples

The examples were prepared as described in the “Processes” section above. Starting materials: Protected amino acid derivatives (compounds of formula (VI)) were purchased from Bachem. The subsequent N-Methoxy-N-methyl derivatives (25) (compounds of formula (II), Intermediates I-XII) were prepared via Method A (see below)

Examples: The Intermediates I-XII (Weinrebamides) were converted into the Examples 1-26 by the treatment with a solution of either n-BuLi (Examples 1-14, 16, 17, 19, 21, 25) or sec-BuLi (Examples 15, 18, 20, 22-24, 26) and the respective heteroaromatic or aromatic compound via Method B and via Method C (see below) (26). The heteroaromatic compounds were purchased from FLUKA or ALDRICH, the bromobenzene was purchased from CLARIANT.

Specific Synthesis and Analytical Information for Starting Materials and Certain Compounds

General Methods

Method A (Conversion of Compound (VI) to Compound (II)):

The N-protected amino acid derivative (compound (VI), 1.0 equiv.) was dissolved in dry THF and cooled to 0° C. To this mixture, HCl.HN(CH₃)OCH₃ (1.05 equiv.), NEt₃ (1.07 equiv.), HOBt (1.1 equiv.), and diisopropylcarbodiimide (1.1 equiv.) were added and the whole mixture was stirred overnight. The solvent was evaporated under reduced pressure. The obtained crude compound was dissolved in EE, washed with 5% aqueous citric acid, water, aqueous NaHCO₃, water and brine and dried over Na₂SO₄. After filtration the solvent was evaporated under reduced pressure. The crude compound was purified by flash chromatography generating Intermediates I-XII.

Method B (Reaction of Compound (II) with Compound (III) to Form Compound (I)):

A stirred solution of the heteroaryl compound (III) (3.0 equiv.) in dry THF was cooled to −78° C. n-BuLi (3.0 equiv.) was added dropwise. After 10 minutes, a solution of the appropriate Intermediate (compound (II), 1.0 equiv.) in dry THF was added dropwise. The mixture was stirred for 2 h at −50° C. before the mixture was diluted with pH-7 buffer solution. The product was extracted with EE. The solvent was dried over Na₂SO₄, filtered and evaporated under reduced pressure. The crude compound was purified by flash chromatography generating the Examples 1, 2, 4, 5, 6, 10, 11, 13, 14, 16, 17, 19, 20, 21, 23, 24, 25, 26

Method C (Reaction of Compound (II) with Compound (III) to Form Compound (I)):

A stirred solution of the heteroaryl or aryl compound (compound (III), (1.0 equiv.) in dry THF was cooled to −78° C. sec-BuLi (3.0 equiv.) was added dropwise. After 10 minutes, a solution of the appropriate Intermediate (compound (II), 3.0 equiv.) in dry THF was added dropwise. The mixture was stirred for 30 minutes before the mixture was diluted with pH-7 buffer solution. The product was extracted with EE. The solvent was dried over Na₂SO₄, filtered and evaporated under reduced pressure. The crude compound was purified by flash chromatography generating the Examples 3, 7, 8, 9, 12, 15, 18, 22.

Intermediates (Exemplary Compounds of Formula (II))

Intermediate I: Cbz-L-Pro-N(CH₃)OCH₃
Intermediate I was prepared according to Method A starting from Cbz-L-Pro-OH, the crude compound was purified by flash chromatography, yield of the purified compound: 76%.

Intermediate II: Cbz-L-Phe-N(CH₃)OCH₃
Intermediate II was prepared according to Method A starting from Cbz-L-Phe-OH, the crude compound was purified by flash chromatography, yield of the purified compound: 75%.

Intermediate III: Boc-L-Pro-N(CH₃)OCH₃
Intermediate III was prepared according to Method A starting from Boc-L-Pro-OH, the crude compound was purified by flash chromatography, yield of the purified compound: 62%.

Intermediate IV: Cbz-L-Ala-N(CH₃)OCH₃
Intermediate IV was prepared according to Method A starting from Cbz-L-Ala-OH, the crude compound was purified by flash chromatography, yield of the purified compound: 89%.

Intermediate V: Boc-L-Leu-N(CH₃)OCH₃
Intermediate V was prepared according to Method A starting from Boc-L-Leu-OH, the crude compound was purified by flash chromatography, yield of the purified compound: 64%.

Intermediate VI: Boc-L-Phe-N(CH₃)OCH₃
Intermediate VI was prepared according to Method A starting from Boc-L-Phe-OH, the crude compound was purified by flash chromatography, yield of the purified compound: 98%.

Intermediate VII: Boc-L-(p-NO₂)Phe-N(CH₃)OCH₃
Intermediate VII was prepared according to Method A starting from Boc-L-(p-NO₂)Phe-OH, the crude compound was purified by flash chromatography, yield of the purified compound: 97%.

Intermediate VIII: Aloc-L-Phe-N(CH₃)OCH₃
Intermediate VIII was prepared according to Method A starting from Aloc-L-Phe-OH, the crude compound was purified by flash chromatography, yield of the purified compound: 81%.

Intermediate IX: Boc-L-Trp-N(CH₃)OCH₃
Intermediate IX was prepared according to Method A starting from Boc-L-Trp-OH, the crude compound was purified by flash chromatography, yield of the purified compound: 96%.

Intermediate X: Cinnamoyl-L-Phe-N(CH₃)OCH₃
Intermediate X was prepared according to Method A starting from Cinnamoyl-L-Phe-OH, the crude compound was purified by flash chromatography, yield of the purified compound: 97%.

Intermediate XI: Cbz-D-Phe-N(CH₃)OCH₃
Intermediate XI was prepared according to Method A starting from Cbz-D-Phe-OH, the crude compound was purified by flash chromatography, yield of the purified compound: 99%.

Intermediate XII: Boc-L-(p-Iodo)Phe-N(CH₃)OCH₃
Intermediate XII was prepared according to Method A starting from Boc-L-(p-Iodo)Phe-OH, the crude compound was purified by flash chromatography, yield of the purified compound: 95%.

### Examples

### Exemplary Compounds of Formula (I) and Similar Compounds)

### Example 1

### 2-[Cbz-L-Pro]Benzothiazole

Example 1 was prepared via Intermediate I and Method B (compound (III): Benzothiazole, yield of the purified compound: 11%).

### Example 2

### 2-[Cbz-L-Phe]Benzothiazole

Example 2 was prepared via Intermediate II and Method B (compound (III): Benzothiazole, yield of the purified compound: 22%).

### Example 3

### 2-[Cbz-L-Phe]Thiophene

Example 3 was prepared via Intermediate II and Method C (compound (III): Thiophene, yield of the purified compound: 63%).

### Example 4

### 2-[Cbz-D-Phe]Thiazole

Example 4 was prepared via Intermediate XI and Method B (compound (III): Thiazole, yield of the purified compound: 76%).

### Example 5

### 2-[Cbz-L-Phe]Thiazole

Example 5 was prepared via Intermediate II and Method B (compound (III) Thiazole, yield of the purified compound: 20%).

### Example 6

### 2-[Cbz-L-Ala]Benzothiazole

Example 6 was prepared via Intermediate IV and Method B (compound (III): Benzothiazole, yield of the purified compound: 44%).

### Example 7

### 2-[Cbz-L-Phe]Pyridine

Example 7 was prepared via Intermediate II and Method C (compound (III): 2-Bromopyridine, yield of the purified compound: 34%).

### Example 8

### 2-[Cbz-L-Phe]Benzo[b]thiophene

Example 8 was prepared via Intermediate II and Method C (compound (III): Benzo[b]thiophene, yield of the purified compound: 6%).

### Example 9

### Cbz-L-Phe-Benzene

Example 9 was prepared via Intermediate II and Method C (compound (III): Bromobenzene, yield of the purified compound: 35%).

### Example 10

### 2-[Cbz-L-Ala]Thiazole

Example 10 was prepared via Intermediate IV and Method B (compound (III) Thiazole, yield of the purified compound: 79%).

### Example 11

### 2-[Boc-L-(p-NO₂)Phe]Thiazole

Example 11 was prepared via Intermediate VII and Method B (compound (III): Thiazole, yield of the purified compound: 42%).

### Example 12

### 2-[Boc-L-(p-NO₂)Phe]Furan

Example 12 was prepared via Intermediate VII and Method C (compound (III): Furan, yield of the purified compound: 8%).

### Example 13

### 2-[Aloc-L-Phe]Benzothiazole

Example 13 was prepared via Intermediate VIII and Method B (compound (III): Benzothiazole, yield of the purified compound: 37%).

### Example 14

### 2-[Aloc-L-Phe]Thiazole

Example 14 was prepared via Intermediate VIII and Method B (compound (III) Thiazole, yield of the purified compound: 45%).

### Example 15

### 2-[Aloc-L-Phe]Furan

Example 15 was prepared via Intermediate VIII and Method C (compound (III): Furan, yield of the purified compound: 76%).

### Example 16

### 2-[Boc-L-Trp]Benzothiazole

Example 16 was prepared via Intermediate IX and Method B (compound (III): Benzothiazole, yield of the purified compound: 10%).

### Example 17

### 2-[Boc-L-Trp]Thiazole

Example 17 was prepared via Intermediate IX and Method B (compound (III): Thiazole, yield of the purified compound: 49%).

### Example 18

### 2-[Boc-L-Trp]Furan

Example 18 was prepared via Intermediate IX and Method C (compound (III): Furan, yield of the purified compound: 27%).

### Example 19

### 2-[Cinnamoyl-L-Phe]Thiazole

Example 19 was prepared via Intermediate X and Method B (compound (III) Thiazole, yield of the purified compound: 50%).

### Example 20

### 2-[Boc-L-Leu]Benzothiazole

Example 20 was prepared via Intermediate V and Method B (compound (III): Benzothiazole, yield of the purified compound: 41%).

### Example 21

### 2-[Boc-L-(p-NO₂)Phe]Benzothiazole

Example 21 was prepared via Intermediate VII and Method B (compound (III): Benzothiazole, yield of the purified compound: 29%).

### Example 22

### 2-[Boc-L-(p-Iodo)Phe]Furan

Example 22 was prepared via Intermediate XII and Method C (compound (III): Furan, yield of the purified compound: 59%).

### Example 23

### 2-[Boc-L-(p-Iodo)Phe]Thiazole

Example 23 was prepared via Intermediate XII and Method B (compound (III): Thiazole, yield of the purified compound: 34%).

### Example 24

### 2-[Boc-L-Pro]Benzothiazole

Example 24 was prepared via Intermediate III and Method B (compound (III) Benzothiazole, yield of the purified compound: 49%).

### Example 25

### 2-[Boc-L-Pro]Thiazole

Example 25 was prepared via Intermediate III and Method B (compound (III): Thiazole, yield of the purified compound: 74%).

### Example 26

### 2-[Boc-L-Phe]Thiazole

Example 26 was prepared via Intermediate VI and Method B (compound (III): Thiazole, yield of the purified compound: 68%).

Abbreviations

Amino Acids

| Amino Acid | Code | Side chain |
|---|---|---|
| Glycine | Gly | H |
| Alanine | Ala | Me |
| Valine | Val | —CH(CH₃)₂ |
| Leucine | Leu | —CH₂CH(CH₃)₂ |
| Isoleucine | Ile | —CH(CH₃)CH₂CH₃ |
| Tyrosine | Tyr | —CH₂(phen-4-ol) |
| Tryptophan | Trp | —CH₂(indol-3-yl) |
| Phenylalanine | Phe | —CH₂Ph |
| Cysteine | Cys | —CH₂SH |
| Methionine | Met | —CH₂CH₂SCH₃ |
| Serine | Ser | —CH₂OH |
| Threonine | Thr | —CH(CH₃)OH |
| Aspartic acid | Asp | —CH₂C(O)OH |
| Glutamic acid | Glu | —CH₂CH₂C(O)OH |
| Asparagine | Asn | —CH₂C(O)NH₂ |
| Glutamine | Gln | —CH₂CH₂C(O)NH₂ |

General Abbreviations

Aloc Allyloxycarbonyl

Boc tert.-Butyloxycarbonyl

n-BuLi n-Butyllithium

sec-BuLi sec-Butyllithium

Cbz Benzyloxycarbonyl

CETP Cholesteryl ester transfer protein

DPP IV Dipeptidyl peptidase IV

EE Ethyl ether

Et Ethyl

HOBt 1-hydroxybenzotriazole

Me Methyl

NEt₃ Triethylamine

PEP prolyl endopeptidase

Ph Phenyl

pNA 4-Nitroanilide

TFA Trifluoroacetic acid

TLC Thin Layer Chromatography

Throughout the specification and the claims which follow, unless the context requires otherwise, the word ‘comprise’, and variations such as ‘comprises’ and ‘comprising’, will be understood to imply the inclusion of a stated integer, step, group of integers or group of steps but not to the exclusion of any other integer, step, group of integers or group of steps.

All patents and patent applications mentioned above are herein incorporated in their entirety by reference.

### REFERENCES

1. Handbook of Proteolytic Enzymes (1998). Barrett, A. J., Rawlings, N. D., and Woessner, J. F. London, Academic Press.
2. Goossens, F., De, M., I, Vanhoof, G., Hendriks, D., Vriend, G., and Scharpe, S. (1995) Eur. J. Biochem. 233, 432-441.
3. Barrett, A. J. and Rawlings, N. D. (1992) Biol. Chem. Hoppe Seyler 373, 353-360.
4. Fulop, V., Bocskei, Z., and Polgar, L. (1998) Cell 94, 161-170.
5. Wetzel, W., Wagner, T., Vogel, D., Demuth, H. U., and Balschun, D. (1997) Neuropeptides 31, 41-45.
6. Demuth, H. U., Neumann, U., and Barth, A. (1989) J. Enzyme Inhib. 2, 239-248.
7. Goossens, F., De, M., I, Vanhoof, G., and Scharpe, S. (1996) Eur. J. Clin. Chem. Clin. Biochem. 34, 17-22.
8. Toide, K., Iwamoto, Y., Fujiwara, T., and Abe, H. (1995) J. Pharmacol. Exp. Ther. 274, 1370-1378.
9. Shinoda, M., Matsuo, A., and Toide, K. (1996) Eur. J. Pharmacol. 305, 31-38.
10. Katsube, N., Sunaga, K., Aishita, H., Chuang, D. M., and Ishitani, R. (1999) J. Pharmacol. Exp. Ther. 288, 6-13.
11. Shishido, Y., Furushiro, M., Tanabe, S., Shibata, S., Hashimoto, S., and Yokokura, T. (1999) Eur. J. Pharmacol. 372, 135-142.
12. Mentlein, R. (1988) FEBS Lett. 234, 251-256.
13. Wilk, S. (1983) Life Sci. 33, 2149-2157.
14. Bennett, G. W., Ballard, T. M., Watson, C. D., and Fone, K. C. (1997) Exp. Gerontol. 32, 451-469.
15. Huston, J. P. and Hasenohrl, R. U. (1995) Behay. Brain Res. 66, 117-127.
16. Liu, X. G. and Sandkuhler, J. (1998) Neuroscience 86, 1209-1216.
17. Abdel-Latif, A. A. (1989) Life Sci. 45, 757-786.
18. Defea, K., Schmidlin, F., Dery, O., Grady, E. F., and Bunnett, N. W. (2000) Biochem. Soc. Trans. 28, 419-426.
19. Voronin, L., Byzov, A., Kleschevnikov, A., Kozhemyakin, M., Kuhnt, U., and Volgushev, M. (1995) Behav. Brain Res. 66, 45-52.
20. Komatsu, Y. (1996) J. Neurosci. 16, 6342-6352.
21. Kimura, A., Yoshida, I., Takagi, N., and Takahashi, T. (1999) J. Biol. Chem. 274, 24047-24053.
22. Hasebe, T., Hua, J., Someya, A., Morain, P., Checker, F., and Nagaoka, I. (2001) J. Leu. Biol. 69, 963-968.
23. Bodanszky, M. and Bodanszky, A. (1994) The practice of peptide synthesis, 2^{nd} Edition; Springer-Verlag: Berlin Heidelberg.
24. Tietze, L. F. and Eicher, Th. (1981) Reaktionen and Synthesen im organisch-chemisches Grundpraktikum; Georg Thieme Verlag Stuttgart.
25. Yasuma, T.; Oi, S.; Choh, N.; Nomura, T.; Furuyama, N.; Nishimura, A.; Fujisawa, Y.; and Sohda, T. (1998) J. Med. Chem. 41, 4301-4308.
26. Joyeau, R., Maoulida, C., Guillet, C., Frappier, F., Teixeira, A. R., Schrevel, J., Santana, J., and Grellier, P. (2000) Eur. J. Med. Chem. 35, 7343-7347.
27. Guo, B., Zhai, D., Cabezas, E., Welsh, K., Nouraini, S., Satterthwait, A. C., and Reed, J. C. (2003) Nature 423, 456-461.
28. Ikonen, M., Liu, B., Hashimoto, Y., Ma, L., Lee, K. W., Niikura, T., Nishimoto, I., and Cohen, P. (2003) Proc. Natl. Acad. Sci. U.S.A. 100, 13042-13047.
29. Sponne, I., Fifre, A., Koziel, V., Kriem, B., Oster, T., and Pillot, T. (2004) Mol. Cell Neurosci. 25, 95-102.
30. Hashimoto, Y., Niikura, T., Tajima, H., Yasukawa, T., Sudo, H., Ito, Y., Kita, Y., Kawasumi, M., Kouyama, K., Doyu, M., Sobue, G., Koide, T., Tsuji, S., Lang, J., Kurokawa, K., and Nishimoto, I. (2001) Proc. Natl. Acad. Sci. U.S.A. 98, 6336-6341
31. Kariya, S., Takahashi, N., Hirano, M., and Ueno, S. (2003) Mol. Cell Biochem. 254, 83-89.
32. Hashimoto, Y., Ito, Y., Niikura, T., Shao, Z., Hata, M., Oyama, F., and Nishimoto, I. (2001) Biochem. Biophys. Res. Commun. 283, 460-468.
33. Tajima, H., Niikura, T., Hashimoto, Y., Ito, Y., Kita, Y., Terashita, K., Yamazaki, K., Koto, A., Aiso, S., and Nishimoto, I. (2002) Neurosci. Lett. 324, 227-231.
34. Hashimoto, Y., Niikura, T., Ito, Y., Sudo, H., Hata, M., Arakawa, E., Abe, Y., Kita, Y., and Nishimoto, I. (2001) J. Neurosci. 21, 9235-9245.
35. Yamagishi, Y., Hashimoto, Y., Niikura, T., and Nishimoto, I. (2003) Peptides 24, 585-595.
36. Piccini, A., Russo, C., Gliozzi, A., Relini, A., Vitali, A., Borghi, R., Giliberto, L., Armirotti, A., D'Arrigo, C., Bachi. A., Cattaneo A., Canale, C., Torrassa, S., Saido, T. C., Markesbery, W., Gambetti, P., Tabaton, M. (2005) J. Biol. Chem. 280, 34186-34192.
37. (2004) Bioorg. Med. Chem. Lett. 14, 5579-5583.
38. (2000) Eur. J. of Med. Chem. 35, 257-266.
39. (1996) Bioorg. Med. Chem. Lett. 6, 3009-3012.
40. (2005) Tetrah. Lett. 46, 8587-8589.
41. (2004) Org. Lett. 6, 4423-4426.
42. (1998) J. Braz. Chem. Soc. 9, 381-387.
43. (1998) Tetrah. Lett. 39, 3189-3192.

1. A compound of formula (I) 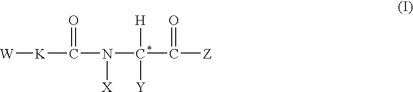
or a pharmaceutically acceptable salt, polymorph or solvate thereof, including all tautomers and stereoisomers thereof,
wherein:
K represents O, S or NH;
W represents —C₁₋₆alkyl-aryl, —C₂₋₆alkenylaryl; —C₁₋₆alkylheteroaryl or —C₂₋₆alkenylheteroaryl;
X represents H or methyl;
Y represents a side chain of an amino acid selected from
Gly; Ala; Val; Leu; Ile; Met; Phe; Ser; Thr; Trp; Asn; Gln; and
the side chain of an analogue of Phe in which the aromatic moiety is substituted by one more groups selected from halogen, nitro, C₁₋₄alkyl, C₁₋₄haloalkyl, hydroxyl, C₁₋₄alkoxy and C₁₋₄haloalkoxy; and
the side chain of an analogue of Ser or Thr in which the hydroxyl group is substituted by C₁₋₆alkyl; and
the side chain of an analogue of Trp in which the heteroaromatic moiety is substituted by one or more C₁₋₄alkyl groups; and
the side chain of an analogue of Cys in which the thiol group is substituted by C₁₋₆alkyl; and
the side chain of an analogue of Asp or Glu wherein the carboxylic acid group has been converted into a C₁₋₆alkyl ester; and
the side chain of an analogue of Asn or Gln wherein the —NH₂ of the amide has been converted into an —NH(C₁₋₄alkyl) or —N(C₁₋₄alkyl)(C₁₋₄alkyl) group; and
the side chain of an analogue of Lys or Arg wherein the —NH₂ of the amine has been converted into an —NHC(O)C₁₋₄alkyl group or an —N(C₁₋₄alkyl)C(O)C₁₋₄alkyl group;
or X and Y are joined such that 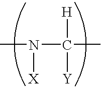
represents 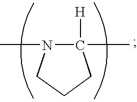
Z represents heteroaryl;
and
when Y represents the side chain of an amino acid selected from Phe; Trp; or
the side chain of an analogue of Phe in which the aromatic moiety is substituted by one more groups selected from halogen, nitro, C₁₋₄alkyl, C₁₋₄haloalkyl, hydroxyl, C₁₋₄alkoxy and C₁₋₄haloalkoxy; or
the side chain of an analogue of Trp in which the heteroaromatic moiety is substituted by one or more C₁₋₄alkyl groups;
then Z can also represent aryl;
wherein any of the aforesaid carbocyclyl and heterocyclyl may be optionally substituted by one or more groups selected from oxo and methyl; and
wherein any of the aforesaid aryl and heteroaryl may optionally substituted by one or more groups selected from: C₁₋₆alkyl, C₂₋₆alkenyl, C₂₋₆alkynyl, C₁₋₆haloalkyl, —C₁₋₆thioalkyl, —SO₂C₁₋₄alkyl, C₁₋₆alkoxy-, —O—C₃₋₈cycloalkyl, C₃₋₈cycloalkyl, —SO₂C₃₋₈cycloalkyl, C₃₋₆alkenyloxy-, C₃₋₆alkynyloxy-, —C(O)C₁₋₆alkyl, C₁₋₆alkoxy-C₁₋₆alkyl-, nitro, halogen, cyano, hydroxyl, —C(O)OH, —NH₂, —NHC₁₋₄alkyl, —N(C₁₋₄alkyl)(C₁₋₄alkyl), —C(O)N(C₁₋₄alkyl)(C₁₋₄alkyl), —C(O)NH₂ and —C(O)NH(C₁₋₄alkyl);
wherein * represents a stereogenic centre;
and wherein the following compounds (a) to (k) are disclaimed from the definition of formula (I):

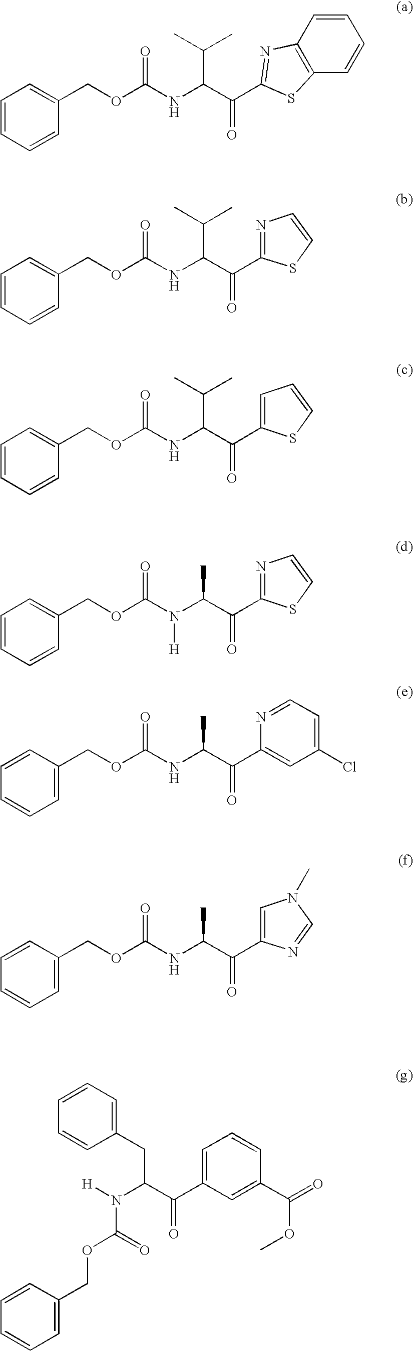

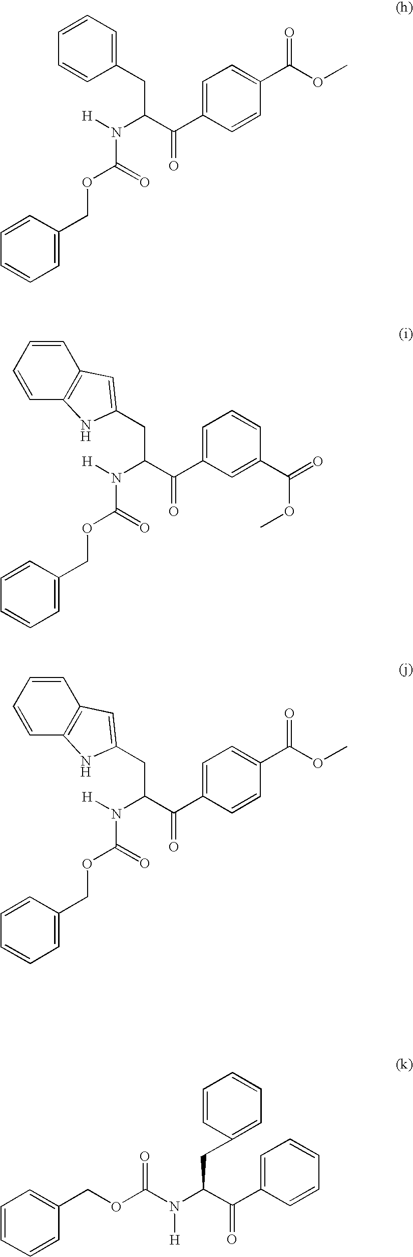

2. A compound of formula (I) according to claim 1 wherein Z represents heteroaryl which may optionally be substituted.

3. A compound of formula (I) according to claim 1 wherein Z represents aryl which may optionally be substituted.

4. A compound of formula (I) according to claim 2 wherein
Z represents pyridinyl or a five membered heteroaryl group containing one or two heteroatoms optionally fused to a phenyl ring wherein any of the aforesaid pyridinyl, heteroaryl or phenyl may optionally be substituted.

5. A compound of formula (I) according to claim 4 wherein
Z represents thiazol-2-yl.

6. A compound of formula (I) according to claim 4 wherein
Z represents benzthiazol-2-yl.

7. A compound of formula (I) according to claim 3 wherein
Z represents optionally substituted phenyl.

8. A compound of formula (I) according to claim 1, wherein K represents O.

9. A compound of formula (I) according to claim 1, wherein
W represents —C₁₋₆alkyl-aryl which aryl may optionally be substituted.

10. A compound of formula (I) according to claim 9 wherein W represents benzyl.

11. A compound of formula (I) according to claim 1, wherein
X represents H and Y represents the side-moiety of Ala, Leu, Trp or Phe or the side moiety of an analogue of Phe in which the aromatic moiety is substituted.

12. A compound of formula (I) according to claim 1, wherein X and Y are joined such that 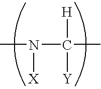
represents

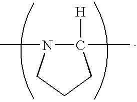

13. A compound of formula (I) according to claim 1, wherein
the stereochemistry at * is the same as that of the naturally occurring L-amino acid or analogue thereof.

14. A compound of formula (I) according to claim 1 which is defined by one of Examples 1 to 8 or a pharmaceutically acceptable salt, polymorph or solvate of any one thereof, including all tautomers and stereoisomers thereof.

15. A compound as defined by one of Examples 11 to 23 or a pharmaceutically acceptable salt, polymorph or solvate of any one thereof, including all tautomers and stereoisomers thereof.

16. A compound of formula (I) 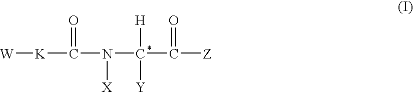
wherein:
K represents O, S or NH;
W represents —C₁₋₆alkyl-aryl, —C₂₋₆alkenylaryl; —C₁₋₆alkylheteroaryl or —C₂₋₆alkenylheteroaryl;
X represents H or methyl;
Y represents a side chain of an amino acid selected from
Gly; Ala; Val; Leu; Ile; Met; Phe; Ser; Thr; Trp; Asn; Gln; and
the side chain of an analogue of Phe in which the aromatic moiety is substituted by one more groups selected from halogen, nitro, C₁₋₄alkyl, C₁₋₄haloalkyl, hydroxyl, C₁₋₄alkoxy and C₁₋₄haloalkoxy; and
the side chain of an analogue of Ser or Thr in which the hydroxyl group is substituted by C₁₋₆alkyl; and
the side chain of an analogue of Trp in which the heteroaromatic moiety is substituted by one or more C₁₋₄alkyl groups; and
the side chain of an analogue of Cys in which the thiol group is substituted by C₁₋₆alkyl; and
the side chain of an analogue of Asp or Glu wherein the carboxylic acid group has been converted into a C₁₋₆alkyl ester; and
the side chain of an analogue of Asn or Gln wherein the —NH₂ of the amide has been converted into an —NH(C₁₋₄alkyl) or —N(C₁₋₄alkyl)(C₁₋₄alkyl) group; and
the side chain of an analogue of Lys or Arg wherein the —NH₂ of the amine has been converted into an —NHC(O)C₁₋₄alkyl group or an —N(C₁₋₄alkyl)C(O)C₁₋₄alkyl group;
or X and Y are joined such that 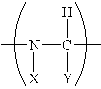
represents 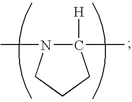
Z represents heteroaryl;
and
when Y represents the side chain of an amino acid selected from
Phe; Trp; and
the side chain of an analogue of Phe in which the aromatic moiety is substituted by one more groups selected from halogen, nitro, C₁₋₄alkyl, C₁₋₄haloalkyl, hydroxyl, C₁₋₄alkoxy and C₁₋₄haloalkoxy; and
the side chain of an analogue of Trp in which the heteroaromatic moiety is substituted by one or more C₁₋₄alkyl groups;
then Z can also represent aryl;
wherein any of the aforesaid carbocyclyl and heterocyclyl may be optionally substituted by one or more groups selected from oxo and methyl; and
wherein any of the aforesaid aryl and heteroaryl may optionally substituted by one or more groups selected from: C₁₋₆alkyl, C₂₋₆alkenyl, C₂₋₆alkynyl, C₁₋₆haloalkyl, —C₁₋₆thioalkyl, —SO₂C₁₋₄alkyl, C₁₋₆alkoxy-, —O—C₃₋₈cycloalkyl, C₃₋₈cycloalkyl, —SO₂C₃₋₈cycloalkyl, C₃₋₆alkenyloxy-, C₃₋₆alkynyloxy-, —C(O)C₁₋₆alkyl, C₁₋₆alkoxy-C₁₋₆alkyl-, nitro, halogen, cyano, hydroxyl, —C(O)OH, —NH₂, —NHC₁₋₄alkyl, —N(C₁₋₄alkyl)(C₁₋₄alkyl), —C(O)N(C₁₋₄alkyl)(C₁₋₄alkyl), —C(O)NH₂ and —C(O)NH(C₁₋₄alkyl);
wherein * represents a stereogenic centre
or
any one of examples 11 to 26
or
a pharmaceutically acceptable salt, polymorph or solvate of any of the above, including all tautomers and stereoisomers thereof
for use as a pharmaceutical.

17. A pharmaceutical composition comprising a compound of formula (I) 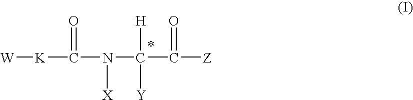
wherein:
K represents O, S or NH;
W represents —C₁₋₆alkyl-aryl, —C₂₋₆alkenylaryl; —C₁₋₆alkylheteroaryl or —C₂₋₆alkenylheteroaryl;
X represents H or methyl;
Y represents a side chain of an amino acid selected from
Gly; Ala; Val; Leu; Ile; Met; Phe; Ser; Thr; Trp; Asn; Gln; and
the side chain of an analogue of Phe in which the aromatic moiety is substituted by one more groups selected from halogen, nitro, C₁₋₄alkyl, C₁₋₄haloalkyl, hydroxyl, C₁₋₄alkoxy and C₁₋₄haloalkoxy; and
the side chain of an analogue of Ser or Thr in which the hydroxyl group is substituted by C₁₋₆alkyl; and
the side chain of an analogue of Trp in which the heteroaromatic moiety is substituted by one or more C₁₋₄alkyl groups; and
the side chain of an analogue of Cys in which the thiol group is substituted by C₁₋₆alkyl; and
the side chain of an analogue of Asp or Glu wherein the carboxylic acid group has been converted into a C₁₋₆alkyl ester; and
the side chain of an analogue of Asn or Gln wherein the —NH₂ of the amide has been converted into an —NH(C₁₋₄alkyl) or —N(C₁₋₄alkyl)(C₁₋₄alkyl) group; and
the side chain of an analogue of Lys or Arg wherein the —NH₂ of the amine has been converted into an —NHC(O)C₁₋₄alkyl group or an —N(C₁₋₄alkyl)C(O)C₁₋₄alkyl group;
or X and Y are joined such that 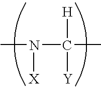
represents 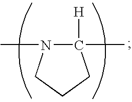
Z represents heteroaryl;
and
when Y represents the side chain of an amino acid selected from
Phe; Trp; and
the side chain of an analogue of Phe in which the aromatic moiety is substituted by one more groups selected from halogen, nitro, C₁₋₄alkyl, C₁₋₄haloalkyl, hydroxyl, C₁₋₄alkoxy and C₁₋₄haloalkoxy; and
the side chain of an analogue of Trp in which the heteroaromatic moiety is substituted by one or more C₁₋₄alkyl groups;
then Z can also represent aryl;
wherein any of the aforesaid carbocyclyl and heterocyclyl may be optionally substituted by one or more groups selected from oxo and methyl; and
wherein any of the aforesaid aryl and heteroaryl may optionally substituted by one or more groups selected from: C₁₋₆alkyl, C₂₋₆alkenyl, C₂₋₆alkynyl, C₁₋₆haloalkyl, —C₁₋₆thioalkyl, —SO₂C₁₋₄alkyl, C₁₋₆alkoxy-, —O—C₃₋₈cycloalkyl, C₃₋₈cycloalkyl, —SO₂C₃₋₈cycloalkyl, C₃₋₆alkenyloxy-, C₃₋₆alkynyloxy-, —C(O)C₁₋₆alkyl, C₁₋₆alkoxy-C₁₋₆alkyl-, nitro, halogen, cyano, hydroxyl, —C(O)OH, —NH₂, —NHC₁₋₄alkyl, —N(C₁₋₄alkyl)(C₁₋₄alkyl), —C(O)N(C₁₋₄alkyl)(C₁₋₄alkyl), —C(O)NH₂ and —C(O)NH(C₁₋₄alkyl);
wherein * represents a stereogenic centre
or
any one of examples 11 to 26
or
a pharmaceutically acceptable salt, polymorph or solvate of any of the above, including all tautomers and stereoisomers thereof
together with one or more therapeutically acceptable diluents or carriers.

18. A compound of formula (I) 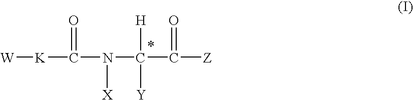
wherein:
K represents O, S or NH;
W represents —C₁₋₆alkyl-aryl, —C₂₋₆alkenylaryl; —C₁₋₆alkylheteroaryl or —C₂₋₆alkenylheteroaryl;
X represents H or methyl;
Y represents a side chain of an amino acid selected from
Gly; Ala; Val; Leu; Ile; Met; Phe; Ser; Thr; Trp; Asn; Gln; and
the side chain of an analogue of Phe in which the aromatic moiety is substituted by one more groups selected from halogen, nitro, C₁₋₄alkyl, C₁₋₄haloalkyl, hydroxyl, C₁₋₄alkoxy and C₁₋₄haloalkoxy; and
the side chain of an analogue of Ser or Thr in which the hydroxyl group is substituted by C₁₋₆alkyl; and
the side chain of an analogue of Trp in which the heteroaromatic moiety is substituted by one or more C₁₋₄alkyl groups; and
the side chain of an analogue of Cys in which the thiol group is substituted by C₁₋₆alkyl; and
the side chain of an analogue of Asp or Glu wherein the carboxylic acid group has been converted into a C₁₋₆alkyl ester; and
the side chain of an analogue of Asn or Gln wherein the —NH₂ of the amide has been converted into an —NH(C₁₋₄alkyl) or —N(C₁₋₄alkyl)(C₁₋₄alkyl) group; and
the side chain of an analogue of Lys or Arg wherein the —NH₂ of the amine has been converted into an —NHC(O)C₁₋₄alkyl group or an —N(C₁₋₄alkyl)C(O)C₁₋₄alkyl group;
or X and Y are joined such that 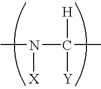
represents 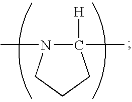
Z represents heteroaryl;
and
when Y represents the side chain of an amino acid selected from
Phe; Trp; and
the side chain of an analogue of Phe in which the aromatic moiety is substituted by one more groups selected from halogen, nitro, C₁₋₄alkyl, C₁₋₄haloalkyl, hydroxyl, C₁₋₄alkoxy and C₁₋₄haloalkoxy; and
the side chain of an analogue of Trp in which the heteroaromatic moiety is substituted by one or more C₁₋₄alkyl groups;
then Z can also represent aryl;
wherein any of the aforesaid carbocyclyl and heterocyclyl may be optionally substituted by one or more groups selected from oxo and methyl; and
wherein any of the aforesaid aryl and heteroaryl may optionally substituted by one or more groups selected from: C₁₋₆alkyl, C₂₋₆alkenyl, C₂₋₆alkynyl, C₁₋₆haloalkyl, —C₁₋₆thioalkyl, —SO₂C₁₋₄alkyl, C₁₋₆alkoxy-, —O—C₃₋₈cycloalkyl, C₃₋₈cycloalkyl, —SO₂C₃₋₈cycloalkyl, C₃₋₆alkenyloxy-, C₃₋₆alkynyloxy-, —C(O)Cl_{—}6alkyl, C₁₋₆alkoxy-C₁₋₆alkyl-, nitro, halogen, cyano, hydroxyl, —C(O)OH, —NH₂, —NHC₁₋₄alkyl, —N(C₁₋₄alkyl)(C₁₋₄alkyl), —C(O)N(C₁₋₄alkyl)(C₁₋₄alkyl), —C(O)NH₂ and —C(O)NH(C₁₋₄alkyl);
wherein * represents a stereogenic centre;
or
any one of examples 11 to 26;
or
a pharmaceutically acceptable salt, polymorph or solvate of any of the above, including all tautomers and stereoisomers thereof
for use in the treatment or prevention of a disease selected from the group consisting of Alzheimer's disease, Down Syndrome, Parkinson disease, Chorea Huntington, pathogenic psychotic conditions, schizophrenia, impaired food intake, sleep-wakefulness, impaired homeostatic regulation of energy metabolism, impaired autonomic function, impaired hormonal balance, impaired regulation, body fluids, hypertension, fever, sleep dysregulation, anorexia, anxiety related disorders including depression, seizures including epilepsy, drug withdrawal and alcoholism, neurodegenerative disorders including cognitive dysfunction, dementia, aphasia, apraxia, agnosia, or any type of amnesias, mild cognitive impairment (MCI), benign forgetfulness and Korsakoff's syndrome, pulmonary vascular disease, restenosis or pulmonary hypertension myocarditis, bronchopulmonary dysplasia, myocardial necrosis or post-cardiac transplant coronary arteriopathy, atherosclerosis, reperfusion injury, hypoxia, ischemia and blood coagulation disorders.

19. A method of treatment or prevention of a disease selected from the group consisting of Alzheimer's disease, Down Syndrome, Parkinson disease, Chorea Huntington, pathogenic psychotic conditions, schizophrenia, impaired food intake, sleep-wakefulness, impaired homeostatic regulation of energy metabolism, impaired autonomic function, impaired hormonal balance, impaired regulation, body fluids, hypertension, fever, sleep dysregulation, anorexia, anxiety related disorders including depression, seizures including epilepsy, drug withdrawal and alcoholism, neurodegenerative disorders including cognitive dysfunction, dementia, aphasia, apraxia, agnosia, or any type of amnesias, mild cognitive impairment (MCI), benign forgetfulness and Korsakoff's syndrome, pulmonary vascular disease, restenosis or pulmonary hypertension myocarditis, bronchopulmonary dysplasia, myocardial necrosis or post-cardiac transplant coronary arteriopathy, atherosclerosis, reperfusion injury, hypoxia, ischemia and blood coagulation disorders which comprises administering to a subject an effective amount of a compound of formula (I) 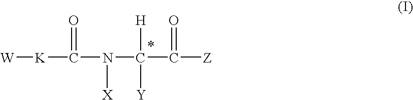
wherein:
K represents O, S or NH;
W represents —C₁₋₆alkyl-aryl, —C₂₋₆alkenylaryl; —C₁₋₆alkylheteroaryl or —C₂₋₆alkenylheteroaryl;
X represents H or methyl;
Y represents a side chain of an amino acid selected from
Gly; Ala; Val; Leu; Ile; Met; Phe; Ser; Thr; Trp; Asn; Gln; and
the side chain of an analogue of Phe in which the aromatic moiety is substituted by one more groups selected from halogen, nitro, C₁₋₄alkyl, C₁₋₄haloalkyl, hydroxyl, C₁₋₄alkoxy and C₁₋₄haloalkoxy; and
the side chain of an analogue of Ser or Thr in which the hydroxyl group is substituted by C₁₋₆alkyl; and
the side chain of an analogue of Trp in which the heteroaromatic moiety is substituted by one or more C₁₋₄alkyl groups; and
the side chain of an analogue of Cys in which the thiol group is substituted by C₁₋₆alkyl; and
the side chain of an analogue of Asp or Glu wherein the carboxylic acid group has been converted into a C₁₋₆alkyl ester; and
the side chain of an analogue of Asn or Gln wherein the —NH₂ of the amide has been converted into an —NH(C₁₋₄alkyl) or —N(C₁₋₄alkyl)(C₁₋₄alkyl) group; and
the side chain of an analogue of Lys or Arg wherein the —NH₂ of the amine has been converted into an —NHC(O)C₁₋₄alkyl group or an —N(C₁₋₄alkyl)C(O)C₁₋₄alkyl group;
or X and Y are joined such that 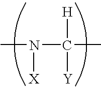
represents 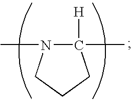
Z represents heteroaryl;
and
when Y represents the side chain of an amino acid selected from
Phe; Trp; and
the side chain of an analogue of Phe in which the aromatic moiety is substituted by one more groups selected from halogen, nitro, C₁₋₄alkyl, C₁₋₄haloalkyl, hydroxyl, C₁₋₄alkoxy and C₁₋₄haloalkoxy; and
the side chain of an analogue of Trp in which the heteroaromatic moiety is substituted by one or more C₁₋₄alkyl groups;
then Z can also represent aryl;
wherein any of the aforesaid carbocyclyl and heterocyclyl may be optionally substituted by one or more groups selected from oxo and methyl; and
wherein any of the aforesaid aryl and heteroaryl may optionally substituted by one or more groups selected from: C₁₋₆alkyl, C₂₋₆alkenyl, C₂₋₆alkynyl, C₁₋₆haloalkyl, —C₁₋₆thioalkyl, —SO₂C₁₋₄alkyl, C₁₋₆alkoxy-, —O—C₃₋₈cycloalkyl, C₃₋₈cycloalkyl, —SO₂C₃₋₈cycloalkyl, C₃₋₆alkenyloxy-, C₃₋₆alkynyloxy-, —C(O)C₁₋₆alkyl, C₁₋₆alkoxy-C₁₋₆alkyl-, nitro, halogen, cyano, hydroxyl, —C(O)OH, —NH₂, —NHC₁₋₄alkyl, —N(C₁₋₄alkyl)(C₁₋₄alkyl), —C(O)N(C₁₋₄alkyl)(C₁₋₄alkyl), —C(O)NH₂ and —C(O)NH(C₁₋₄alkyl);
wherein * represents a stereogenic centre;
or
any one of examples 11 to 26
or
a pharmaceutically acceptable salt, polymorph or solvate of any of the above, including all tautomers and stereoisomers thereof.

20. Use of compound of formula (I) 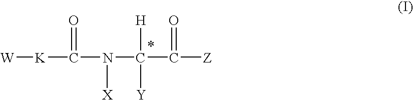
wherein:
K represents O, S or NH;
W represents —C₁₋₆alkyl-aryl, —C₂₋₆alkenylaryl; —C₁₋₆alkylheteroaryl or —C₂₋₆alkenylheteroaryl;
X represents H or methyl;
Y represents a side chain of an amino acid selected from
Gly; Ala; Val; Leu; Ile; Met; Phe; Ser; Thr; Trp; Asn; Gln; and
the side chain of an analogue of Phe in which the aromatic moiety is substituted by one more groups selected from halogen, nitro, C₁₋₄alkyl, C₁₋₄haloalkyl, hydroxyl, C₁₋₄alkoxy and C₁₋₄haloalkoxy; and
the side chain of an analogue of Ser or Thr in which the hydroxyl group is substituted by C₁₋₆alkyl; and
the side chain of an analogue of Trp in which the heteroaromatic moiety is substituted by one or more C₁₋₄alkyl groups; and
the side chain of an analogue of Cys in which the thiol group is substituted by C₁₋₆alkyl; and
the side chain of an analogue of Asp or Glu wherein the carboxylic acid group has been converted into a C₁₋₆alkyl ester; and
the side chain of an analogue of Asn or Gln wherein the —NH₂ of the amide has been converted into an —NH(C₁₋₄alkyl) or —N(C₁₋₄alkyl)(C₁₋₄alkyl) group; and
the side chain of an analogue of Lys or Arg wherein the —NH₂ of the amine has been converted into an —NHC(O)C₁₋₄alkyl group or an —N(C₁₋₄alkyl)C(O)Cl_{—}4alkyl group;
or X and Y are joined such that 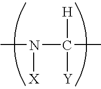
represents 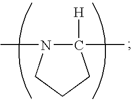
Z represents heteroaryl;
and
when Y represents the side chain of an amino acid selected from
Phe; Trp; and
the side chain of an analogue of Phe in which the aromatic moiety is substituted by one more groups selected from halogen, nitro, C₁₋₄alkyl, C₁₋₄haloalkyl, hydroxyl, C₁₋₄alkoxy and C₁₋₄haloalkoxy; and
the side chain of an analogue of Trp in which the heteroaromatic moiety is substituted by one or more C₁₋₄alkyl groups;
then Z can also represent aryl;
wherein any of the aforesaid carbocyclyl and heterocyclyl may be optionally substituted by one or more groups selected from oxo and methyl; and
wherein any of the aforesaid aryl and heteroaryl may optionally substituted by one or more groups selected from: C₁₋₆alkyl, C₂₋₆alkenyl, C₂₋₆alkynyl, C₁₋₆haloalkyl, —C₁₋₆thioalkyl, —SO₂C₁₋₄alkyl, C₁₋₆alkoxy-, —O—C₃₋₈cycloalkyl, C₃₋₈cycloalkyl, —SO₂C₃₋₈cycloalkyl, C₃₋₆alkenyloxy-, C₃₋₆alkynyloxy-, —C(O)C₁₋₆alkyl, C₁₋₆alkoxy-C₁₋₆alkyl-, nitro, halogen, cyano, hydroxyl, —C(O)OH, —NH₂, —NHC₁₋₄alkyl, —N(C₁₋₄alkyl)(C₁₋₄alkyl), —C(O)N(C₁₋₄alkyl)(C₁₋₄alkyl), —C(O)NH₂ and —C(O)NH(C₁₋₄alkyl);
wherein * represents a stereogenic centre;
or
any one of examples 11 to 26;
or
a pharmaceutically acceptable salt, polymorph or solvate of any of the above, including all tautomers and stereoisomers thereof
in the manufacture of a medicament for the treatment of a disease selected from the group consisting of Alzheimer's disease, Down Syndrome, Parkinson disease, Chorea Huntington, pathogenic psychotic conditions, schizophrenia, impaired food intake, sleep-wakefulness, impaired homeostatic regulation of energy metabolism, impaired autonomic function, impaired hormonal balance, impaired regulation, body fluids, hypertension, fever, sleep dysregulation, anorexia, anxiety related disorders including depression, seizures including epilepsy, drug withdrawal and alcoholism, neurodegenerative disorders including cognitive dysfunction, dementia, aphasia, apraxia, agnosia, or any type of amnesias, mild cognitive impairment (MCI), benign forgetfulness and Korsakoff's syndrome, pulmonary vascular disease, restenosis or pulmonary hypertension myocarditis, bronchopulmonary dysplasia, myocardial necrosis or post-cardiac transplant coronary arteriopathy, atherosclerosis, reperfusion injury, hypoxia, ischemia and blood coagulation disorders.

21. A process for preparation of a compound of formula (I) according to claim 1, which comprises reaction of a compound of formula (II) 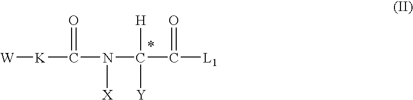
wherein W, K, X and Y are defined as above and L₁ represents a group with a compound of formula (III)
L₂-Z (III)
or a protected derivative thereof wherein Z is defined as above and L₂ represents an appropriate group for the metallation.

22. A compound, use, pharmaceutical composition, method or process according to claim 16, wherein the compound is defined by one of Examples 1 to 26 or a pharmaceutically acceptable salt, polymorph or solvate of any one thereof, including all tautomers and stereoisomers thereof.

23. A compound, use, pharmaceutical composition, method or process according to claim 1, wherein the compound is Example 1 or a pharmaceutically acceptable salt, polymorph or solvate thereof, including all tautomers and stereoisomers thereof.

24. A compound, use, pharmaceutical composition, method or process according to claim 16, wherein the compound is Example 5 or a pharmaceutically acceptable salt, polymorph or solvate thereof, including all tautomers and stereoisomers thereof.
